(19) United States
(12) Patent Application Publication
(10) Pub. No.: US 2024/0108577 A1
(43) Pub. Date: Apr. 4, 2024

(54) CONTROLLED RELEASE OF SUBSTANCES FROM A SOURCE MATERIAL

(71) Applicant: Syqe Medical Ltd., Tel-Aviv (IL)

(72) Inventors: Ilana ZAKS BOHBOT, ModiIn (IL); Joshua AVIRAM, Tirat Carmel (IL); Asaf KROLL, Hofit (IL); Anna FRENKLAKH, Holon (IL); Roee LIFSHITZ, Moshav Beit Hillel (IL); Daniella ATZMONY, Modiin-Maccabim-Reut (IL)

(73) Assignee: Syqe Medical Ltd., Tel-Aviv (IL)

(21) Appl. No.: 18/378,743

(22) Filed: Oct. 11, 2023

Related U.S. Application Data
(63) Continuation-in-part of application No. PCT/IL2023/050370, filed on Apr. 4, 2023.
(60) Provisional application No. 63/327,393, filed on Apr. 5, 2022.

(51) Int. Cl. A61K 9/00; A61K 31/00

(52) U.S. Cl. CPC A61K 9/0078; A61K 31/658

(57) ABSTRACT

This application relates to methods of delivering via inhalation from a source material both pharmacologically-effective doses and placebo doses

### RELATED APPLICATIONS

This application is a Continuation-In-Part (CIP) of PCT Patent Application No. PCT/IL2023/050370 having the International filing date of Apr. 4, 2023 which claims the benefit of priority under 35 USC § 119(e) of U.S. Provisional Patent Application No. 63/327,393 filed on Apr. 5, 2022. The contents of the above applications are all incorporated by reference as if fully set forth herein in their entirety.

### FIELD AND BACKGROUND OF THE INVENTION

The present invention, in some embodiments thereof, relates to a controlled release of substances from source material.

The present invention, in some embodiments thereof, also relates to selective delivery of pharmacologically effective doses and placebo doses of substances released from a same source material.

A paper publication titled “Metabolic Profiling of Cannabis Secondary Metabolites for Evaluation of Optimal Postharvest Storage Conditions”, Front. Plant Sci., 15 Oct. 2020, discloses “The therapeutic use of medical Cannabis is growing, and so is the need for standardized and therapeutically stable Cannabis products for patients. The therapeutic effects of Cannabis largely depend on the content of its pharmacologically active secondary metabolites and their interactions, mainly terpenoids and phytocannabinoids. Once harvested and during storage, these natural compounds may decarboxylate, oxidize, isomerize, react photochemically, evaporate and more. Despite its widespread and increasing use, however, data on the stability of most of the plant's terpenoids and phytocannabinoids during storage is scarce. In this study, we therefore aimed to determine postharvest optimal storage conditions for preserving the composition of naturally biosynthesized secondary metabolites in Cannabis inflorescences and Cannabis extracts. To this end, Cannabis inflorescences (whole versus ground samples) and Cannabis extracts (dissolved in different solvents) from (−)-Δ9-trans-tetrahydrocannabinol- or cannabidiol-rich chemovars, were stored in the dark at various temperatures (25, 4, −30 and −80° C.), and their phytocannabinoid and terpenoid profiles were analyzed over the course of 1 year. We found that in both Cannabis inflorescences and extracts, a storage temperature of 25° C. led to the largest changes in the concentrations of the natural phytocannabinoids over time, making this the most unfavorable temperature compared with all others examined here. Olive oil was found to be the best vehicle for preserving the natural phytocannabinoid composition of the extracts. Terpenoid concentrations were found to decrease rapidly under all storage conditions, but temperatures lower than ˜20° C. and grinding of the inflorescences were the least favorable conditions. Overall, our conclusions point that storage of whole inflorescences and extracts dissolved in olive oil, at 4° C., were the optimal postharvest conditions for Cannabis”.

A paper publication titled “Quality Control of Traditional Cannabis Tinctures: Pattern, Markers, and Stability”, Sci. Pharm. 2016, 84, 567-584; doi:10.3390/scipharm84030567, discloses “Traditional tinctures of Cannabis sativa L. became obsolete before elucidation of the main cannabinoids and routine quality testing for medicines. In view of increasing medicinal use of cannabinoids and associated safety concerns, tinctures from a Δ9-tetrahydrocannabinol (THC)-type chemovar were studied. High-performance liquid chromatography with diode-array detection (HPLC/DAD) was used to determine THC, Δ9-tetrahydrocannabinolic acid A (THCA), cannabinol (CBN), cannabidiol (CBD), cannabidiolic acid (CBDA), cannabigerol (CBG), cannabigerolic acid (CBGA), cannflavin A/B, and total phenolics. Derived group and ratio markers describe absolute and relative profiles when varying plant part (flos, folium), extraction solvent (EtOH percentage), storage conditions (‘shelf’ or ‘fridge’ up to 15 months), and pasteurization (2 h 70° C., 20 min 80° C.). Tinctures from female flowering tops contained ten-fold more cannabinoids than tinctures from leaves; tinctures (80%-90% EtOH) contained ten-fold more cannabinoids than tinctures (40% EtOH). The analysis of CBGA+CBG, the main co-cannabinoids aside from THCA+THC, appears more relevant than CBDA+CBD. The decarboxylation of THCA to THC—the main change during storage of freshly prepared tinctures—is after 15 months in the ‘fridge’ comparable to 3 months on the ‘shelf’. Minimally increased CBN totals did not correlate to diminished totals of THCA and THC (up to 15% after 3 months ‘shelf’, 45% after 15 months ‘fridge’). Instead, total cannabinoids or acidic/neutral cannabinoid ratios are better stability markers. Moderate changes after pasteurization and partial losses below 10% for total cannabinoids after 9 months ‘fridge’ indicate possibilities for a reasonable shelf life. Yet storage and use of non-stabilized tinctures remain critical without authorized specification and stability data because a consistent cannabinoid content is not guaranteed”.

A paper publication titled “The role of time and storage conditions on the composition of hashish and marijuana samples: A four-year study, “Forensic Science International”, discloses the following: “The aim of this study was to investigate the role of time and different real-life storage conditions on the composition of different varieties of cannabis products (hashish and marijuana). Six high-potency cannabis products constituted by herbal and resin materials containing different initial concentrations of delta 9-Tetrahydrocannabinol (THC) were employed for this study. Four representative samples were collected from each study material and were maintained for a prolonged time (four years) under different controlled storage conditions: (A) light (24 h) and room temperature (22 C); (B) darkness (24 h) and room temperature; (C) darkness and refrigeration (4 C); (D) darkness and freezing (−20 C). The concentration of the three main cannabinoids, i.e. THC, Cannabinol (CBN, produced from the degradation of THC), and Cannabidiol (CBD), were measured by GC-FID around every 100 days along the four-year study. Significant changes in the THC (degradation) and CBN (formation) content were detected under storage conditions A and B, and almost 100% of THC was degraded after four years. A mono-exponential function was able to well fit both THC degradation and CBN formation, suggesting that these processes occur with a first order kinetics. Data treatment indicated that the storage temperature and light exposure had two different effects on the conversion of THC to CBN: temperature changed only the speed, light changed both the speed and the stoichiometry of this conversion. Models were proposed which allow to predict the storage time, if unknown, and the initial content of THC (i.e. the concentration of THC at the starting storage time), from the measurement of THC and CBN content at any time under storage condition A. Values predicted are more uncertain at larger storage times and have an accuracy of around 5-10%. These models were also tested on data reported in the literature, and can represent a starting point for further improvements. Prediction models may be helpful for forensic purposes, if the initial concentration of THC or the approximate age of a degraded material need to be estimated, or to plan the storage of delicate samples which need to be re-examined over time”.

### SUMMARY OF THE INVENTION

According to an aspect of some embodiments there is provided a source material cartridge for use with a personal inhaler device, the cartridge comprising:
a source material stored within a housing of the cartridge;
one or more sensors positioned and configured for sensing conditions associated with storage of the cartridge; and
a logger configured to record the data sensed by the one or more sensors.

In some embodiments, the one or more sensors are positioned and configured for sensing one or more of: a temperature outside the housing, a temperature inside the housing, humidity outside the housing, humidity inside the housing, an extent of exposure of the cartridge to light, an extent of rattling of the cartridge.

In some embodiments, a volume of the cartridge is smaller than 60 cm\hat{ }3, and a weight of the cartridge is smaller than 40 grams.

In some embodiments, the logger is configured to record the data up until and/or during use of the cartridge with the inhaler device.

In some embodiments, the housing comprises an RFID label and wherein the logger is incorporated in the RFID label.

In some embodiments, the source material is stored as separate multiple units contained inside the housing, each of the multiple units configured for individual use in the inhaler device.

In some embodiments, the cartridge further comprises a communication module, configured to transfer the recorded data to a controller located externally to the cartridge.

According to an aspect of some embodiments there is provided an inhaler device for use with a source material cartridge, the source material cartridge comprising a logger on which data regarding conditions associated with storage of the cartridge is recorded, the inhaler device comprising:
a heating assembly configured to heat the source material of the cartridge when the cartridge is received by the inhaler device;
a reader positioned and configured to read the data from the logger of the cartridge when the cartridge is received by the inhaler device; and
a controller configured to:
   select a heating profile for the heating assembly according to the data read by the reader; and
   control the heating such that a predetermined amount of at least one substance is released from the source material.

In some embodiments, the inhaler device comprises an airflow path for conducting airflow through the source material when the cartridge is received by the inhaler device.

In some embodiments, the controller is configured to control the heating by controlling a profile of air flowing through the airflow path.

In some embodiments, the controller is configured to control the heating by setting a target temperature or range.

In some embodiments, the controller is configured to control the heating by setting at least one of a duration of heating and a rate of heating.

In some embodiments, the controller is configured to control the heating by setting a target voltage to be applied to the heating assembly.

In some embodiments, the controller is configured to control release of the predetermined amount of at least one substance by heating a selected portion or amount of the source material.

In some embodiments, the controller is preprogrammed with or is configured to calculate or refer to a database or lookup table tying the data pertaining to storage conditions with a plurality of different heating profiles suitable to release the predetermined amount of the at least one substance from source material stored under different conditions.

In some embodiments, the controller is configured to control the airflow profile by controlling one or more of: a rate of airflow through the source material, a target volume of air, a duration of airflow.

In some embodiments, the inhaler device further comprises one or more valves positioned and configured for regulating the airflow through the airflow path, wherein the controller is configured to control actuation of the one or more valves based on the selected airflow profile.

In some embodiments, the heating profile takes into account natural degradation of the source material over time.

According to an aspect of some embodiments there is provided a method of delivering via inhalation at least one substance released from a source material, comprising:
recording, over time, data regarding conditions associated with storage of the source material;
receiving the data at a controller;
selecting, in accordance with the data, a heating profile for the source material; and
applying the heating profile to release a predetermined amount of at least one substance from the source material.

In some embodiments, the method comprises selecting a profile for airflow passing through the source material.

In some embodiments, the method comprises selecting an amount or a defined portion of the source material to be heated.

In some embodiments, selecting takes into account natural degradation of the source material over time.

In some embodiments, selecting includes calculating natural degradation of the source material based on the recorded data

In some embodiments, the source material comprises cannabis and wherein the at least one substance comprises THC.

In some embodiments, the method comprises obtaining via one or more sensors the data regarding conditions associated with storage.

In some embodiments, the source material is stored in a cartridge sized and configured for individual use with an inhaler device.

In some embodiments, recording is performed from manufacturing of the cartridge and until use of the cartridge in the inhaler device or until removal of the cartridge, following use, from the inhaler device.

In some embodiments, the controller is configured as part of the inhaler device and receiving the data is while the cartridge is operably coupled to the inhaler device.

In some embodiments, selecting is automatically performed at the controller each time a user activates the inhaler device for inhaling through it.

According to an aspect of some embodiments there is provided a method of delivering via inhalation at least one substance released from a source material, comprising:
receiving at a controller data regarding conditions associated with storage of the source material recorded over time;
selecting, in accordance with the data, at least one of a heating profile and an airflow profile for the source material; and
applying at least one of the heating profile and the airflow profile to release a predetermined amount of at least one substance from the source material.

In some embodiments, the method comprises selecting a profile for airflow passing through the source material.

In some embodiments, selecting takes into account natural degradation of the source material over time.

In some embodiments, selecting includes calculating natural degradation of the source material based on the recorded data.

In some embodiments, the source material comprises cannabis and wherein the at least one substance comprises THC.

In some embodiments, the source material is stored in a cartridge sized and configured for individual use with an inhaler device.

In some embodiments, the controller is configured as part of the inhaler device and receiving the data is while the cartridge is operably coupled to the inhaler device.

In some embodiments, selecting is automatically performed at the controller each time a user activates the inhaler device for inhaling through it.

According to an aspect of some embodiments there is provided a method of delivering via inhalation at least one substance released from a source material, comprising:
recording, over time, data regarding conditions associated with storage of the source material;
receiving the data at a controller;
selecting, in accordance with the data, an operation profile, the operation profile including one or more of: a heating profile, an airflow profile and an amount of source material to be heated;
and applying the operation profile to release a predetermined amount of at least one substance from the source material.

According to an aspect of some embodiments there is provided a method of selectively delivering via inhalation both clinically effective doses and clinically ineffective doses released from a same source material, comprising:
scheduling a delivery regimen including a plurality of clinically effective doses and a plurality of clinically ineffective doses serving as placebo;
based on the type of dose to be delivered, between clinically effective and clinically ineffective, selecting at least one of a heating profile and an airflow profile to be applied to the source material;
releasing from the source material one or more substances of a type and/or amount which is either clinically effective or clinically ineffective, depending on the type of dose to be delivered.

In some embodiments, the clinically ineffective dose comprises substances having a taste and/or scent similar or close to those of substances released for a clinically effective dose.

In some embodiments, the clinically ineffective dose further comprises the release of vapor.

In some embodiments, a clinically ineffective dose includes releasing one or more sensory substances from the source material, while avoiding or reducing release of active substances from the source material.

In some embodiments, the source material comprises cannabis, wherein the one or more sensory substances include terpenes and the active substances include THC.

In some embodiments, selecting of a heating profile comprises selecting a temperature range which is low enough so that only the one or more sensory substances are vaporized from the source material, while one or more active substances do not reach a vaporization state.

In some embodiments, the method further comprises tracking conditions associated with storage of the source material over time, and wherein selecting is performed at least partially based on the tracked conditions.

According to an aspect of some embodiments there is provided a method of delivering via inhalation at least one clinically ineffective dose from ground cannabis inflorescence, comprising applying heating to the ground cannabis inflorescence; wherein a profile of the heating is selected to heat the ground cannabis inflorescence to a target temperature between 60° C. and 130° C.

In some embodiments, the method comprises releasing a plurality of terpenes from the ground cannabis inflorescence and delivering to a user via inhalation.

In some embodiments, the method comprises reducing or avoiding release of THC from the ground cannabis inflorescence.

According to an aspect of some embodiments there is provided a method of releasing THC from ground cannabis inflorescence which had been stored over time, comprising:
tracking, over time, a storage temperature and a storage humidity level for the ground cannabis inflorescence;
controlling heating of the ground cannabis inflorescence according to the tracked temperature and humidity to release a predetermined amount of THC from the ground cannabis inflorescence.

According to an aspect of some embodiments there is provided a method of delivering via inhalation one or more substances released from a source material, the one or more substances having a selected sensory profile which is experienced by an inhaling user, the method comprising:
controlling heating of the source material to release the one or more substances from the source material; and
delivering the released one or more substances to the inhaling user such that the user experiences the selected sensory profile.

In some embodiments, the sensory profile comprises a flavor and a scent.

In some embodiments, the sensory profile further comprises vapor related sensations or appearance of vapor.

In some embodiments, the one or more substances are clinically ineffective and wherein the selected sensory profile is similar to that experienced when one or more clinically effective substances are released from the same source material.

In some embodiments, the one more clinically ineffective substances include terpenes and the one more clinically effective substances include cannabinoids.

According to an aspect of some embodiments, the present invention provides an inhaler for delivery of types of doses, including pharmacologically effective dose and a placebo dose, to a user by inhalation from the same source material, the inhaler comprising:
an airflow conduit for conducting airflow to a proximal opening of a mouthpiece;
a holder configured to position a source material (such as a source material dose unit) at a delivery position within the airflow conduit; and
a circuitry programmed to apply heating and/or airflow profiles to the source material suitable for the delivery of the selected type of dose.

In some embodiments the placebo type of dose comprises a sensory profile, the sensory profile may include one or more of: release of a sensory substance, conducting an airflow pattern and/or releasing a vapor.

In some embodiments, the inhaler device circuitry (such as a controller of the device) is preprogrammed with heating and/or airflow profiles suitable to release selected substance(s) at selected amount(s) according to the selected dose type, either of pharmacologically effective and placebo dose types. Optionally, a heating profile for releasing certain sensory substances while avoiding or reducing the delivery of active substances may be set to reach a low enough temperature range in which only (or mostly) the sensory substances are released from the source material, while the active substances do not reach their vaporization point. In some embodiments a heating profile is designed to reach a target temperature in which at least one selected active substance does not reach its vaporization point, while at least one sensory substance is released, such as by reaching the sensory substance's vaporization point.

In some embodiments, the inhaler device circuitry (such as a controller of the device) is preprogrammed for a placebo dose with heating to a temperature of less than 130° C., less than 120° C., less than 110° C., less than 100° C., less than 90° C., less than 80° C., less than 70° C., less than 60° C., less than 50° C., less than 40° C., less than 30° C.

In some embodiments, the inhaler device circuitry (such as a controller of the device) is preprogrammed for a placebo dose with no heating, and having an airflow profile suitable to release selected substance(s) at selected amount(s) according to a delivery regimen. In some embodiments, the airflow profile includes controlling airflow valves to allow air to be drawn through the source material at interchangeable flowrates. In some embodiments, patterns of airflow comprising interchangeable flow rates are applied through the source material. In some embodiments patterns of airflow in combination with the release of sensory substance provides a sensory profile. In some embodiments patterns of airflow in combination with the release of sensory substance while no heat is applied to the source material provides a sensory profile.

According to another aspect of the invention, there is provided a method of delivering via inhalation from a same source material both pharmacologically-effective doses and placebo doses released from an inhalation device, comprising:
selecting a type of dose to be delivered via an inhalation device, the type of dose being a pharmacologically-effective dose or a placebo dose;
based on the type of dose, selecting at least one of a heating profile and an airflow profile to be applied to the source material;
releasing from the source material one or more substances of a type and/or amount which is either pharmacologically effective or psychologically effective, depending on the type of dose to be delivered.

According to still another aspect, there is provided a method of delivering via inhalation both pharmacologically-effective doses and placebo doses released from an inhalation device comprising a source material, comprising:
selecting a type of dose to be delivered via an inhalation device, the type of dose being a pharmacologically-effective dose or a placebo dose;
based on the type of dose, selecting at least one of a heating profile and an airflow profile to be applied to the source material;
releasing from the source material one or more substances of a type and/or amount which is either pharmacologically effective or psychologically effective, depending on the type of dose to be delivered.

In some embodiments, the placebo dose comprises substances having a taste and/or scent similar or close to those of substances released for a pharmacologically-effective dose.

In some embodiments, the placebo dose further comprises the release of vapor.

In some embodiments, the releasing a placebo dose includes releasing one or more sensory substances from the source material, while avoiding or reducing release of active substances from the source material.

In some embodiments, the source material comprises cannabis, wherein the one or more sensory substances include terpenes and the active substances include THC.

According to one embodiment, the selecting of a heating profile comprises selecting a temperature range which is low enough so that only the one or more sensory substances are vaporized from the source material, while one or more active substances do not reach a vaporization state.

According to another embodiment, the method further comprises tracking conditions associated with storage of the source material over time, and wherein selecting is performed at least partially based on the tracked conditions.

According to still another aspect of the invention, there is provided a method of delivering via inhalation at least one placebo dose from a THC-carrying material, comprising vaporizing at least one component of the THC-carrying material, wherein the at least one component is not THC; wherein the THC-carrying material is at a temperature below 130° C.

According to some embodiments, the at least one component comprises a terpene.

According to some embodiments, the THC-carrying material is at a temperature below 50° C.

According to some embodiments, the THC-carrying material is at room temperature.

According to still another aspect of the invention, there is provided a method of delivering via inhalation one or more substances released from a source material, the one or more substances having a selected sensory profile which is experienced by an inhaling user, the method comprising: controlling heating of the source material to release the one or more substances from the source material, wherein the source material is maintained at a temperature below 130° C.; and delivering the released one or more substances to the inhaling user such that the user experiences the selected sensory profile.

According to some embodiments, the sensory profile comprises a flavor and a scent.

According to some embodiments, the sensory profile further comprises vapor related sensations or appearance of vapor.

According to some embodiments, the one or more substances are psychologically effective and not pharmacologically effective and wherein the selected sensory profile is similar to that experienced when one or more pharmacologically effective substances are released from the same source material.

According to some embodiments, the one more psychologically effective substances include terpenes and the one more pharmacologically effective substances include cannabinoids.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, some embodiments of methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of some embodiments of the method and/or system, several selected tasks could be implemented by hardware, by software or by firmware or by a combination thereof using an operating system.

For example, hardware for performing selected tasks according to some embodiments could be implemented as a chip or a circuit. As software, selected tasks according to some embodiments could be implemented as a plurality of software instructions being executed by a computer using any suitable operating system. In some embodiments, one or more tasks according to some embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments are herein described, by way of example only, with reference to the accompanying drawings. With specific reference now to the drawings in detail, it is stressed that the particulars shown are by way of example and for purposes of illustrative discussion of embodiments. In this regard, the description taken with the drawings makes apparent to those skilled in the art how embodiments may be practiced.

In the drawings:

FIG. 1 is a flowchart of a method for tracking conditions of a source material over time, and controlling the release of at least one active substance from the source material in accordance with the tracked conditions, according to some embodiments;

FIG. 2 is a schematic diagram of an inhaler system for controlled release of at least one substance from a source material, the system configured for sensing and/or recording conditions of a source material, according to some embodiments;

FIG. 3 is an example of a source material cartridge for use with an inhaler device, the source material cartridge comprising a logger for recording condition data, according to some embodiments;

FIGS. 4A-B are flowcharts of methods for balancing release of pharmacologically effective substances or doses and placebo substances or doses (placebo doses) from a source material, according to some embodiments;

FIG. 5 is a flowchart of a method for sensing and recording conditions of a source material over time, and selecting a heating profile for the source material so as to selectively release placebo doses, according to some embodiments;

FIGS. 6A-B are vaporization plots showing an example of release of THC and various terpenes from cannabis, according to some embodiments;

FIG. 6C is a flowchart of a method for releasing substance(s) having a selected sensory profile (e.g. flavor, scent, appearance), according to some embodiments;

FIGS. 7A-E show results and analysis of an experiment performed by the inventors of this application in which changes in substance compositions were assessed for packaged cannabis stored over time under different environmental conditions, according to some embodiments.

FIG. 7A: U-HPLC analysis of the Bedrocan cultivar. From left to right, the analyzed phytocannabinoids were CBDA, CBGA, CBD, CBN, THC and THCA.

FIG. 7B: Data is reported with the middle circle representing mean value of a plurality of duplicates and the upper and lower lines represent the upper and lower 95% confidence interval values; BL, Baseline; M, Month; LOD, Loss of dry; CBD, Cannabidiol; CBDA, cannabidiol acid; THC, (−)-Δ⁹-trans-tetrahydrocannabinol; THCA, (−)-Δ⁹-trans-tetrahydrocannabinol acid; CBGA, cannabigerolic acid; CBN, cannabinol; Total represent the calculation of the acid and neutral components combined. Statistically significant differences between times of storage were calculated by one-way ANOVA, utilizing Fisher's exact test analyses (whenever significant, stated as the p-value and when not significant stated as N.S.), followed by a Tukey post hoc multiple comparison test (*, p<0.05; **, p<0.01; ***, p<0.001).

FIG. 7C: Data is reported with the middle circle representing mean value and the upper and lower lines represent the upper and lower 95% confidence interval values; BL, Baseline; M, Month; LOD, Loss of dry; CBD, Cannabidiol; CBDA, cannabidiol acid; THC, (−)-Δ⁹-trans-tetrahydrocannabinol; THCA, (−)-Δ⁹-trans-tetrahydrocannabinol acid; CBGA, cannabigerolic acid; CBN, cannabinol; Total represent the calculation of the acid and the neutral components. Statistically significant differences between times of storage were calculated by one-way ANOVA, utilizing Fisher's exact test analyses (whenever significant, stated as the p-value and when not significant stated as N.S.), followed by a Tukey post hoc multiple comparison test (*, p<0.05; **, p<0.01; ***, p<0.001).

FIG. 7D: Data is reported with the middle circle representing mean value and the upper and lower lines represent the upper and lower 95% confidence interval values; BL, Baseline; M, Month; LOD, Loss of dry; CBD, Cannabidiol; CBDA, cannabidiol acid; THC, (−)-Δ⁹-trans-tetrahydrocannabinol; THCA, (−)-Δ⁹-trans-tetrahydrocannabinol; Statistically significant differences between times of storage were calculated by one-way ANOVA, utilizing Fisher's exact test analyses (whenever significant, stated as the p-value and when not significant stated as N.S.), followed by a Tukey post hoc multiple comparison test (*, p<0.05; **, p<0.01; ***, p<0.001).

FIG. 7E: Data is reported with the middle circle representing mean value and the upper and lower lines represent the upper and lower 95% confidence interval values; BL, Baseline; M, Month; LOD, Loss of dry; THC, (−)-Δ⁹-trans-tetrahydrocannabinol; THCA, (−)-Δ⁹-trans-tetrahydrocannabinol acid; CBGA, cannabigerolic acid; CBN, cannabinol; Total represent the calculation of the acid and the neutral components. Statistically significant differences between times of storage were calculated by one-way ANOVA, utilizing Fisher's exact test analyses (whenever significant, stated as the p-value and when not significant stated as N.S.), followed by a Tukey post hoc multiple comparison test (*, p<0.05; **, p<0.01; ***, p<0.001).

### DESCRIPTION OF SPECIFIC EMBODIMENTS OF THE INVENTION

The present invention, in some embodiments thereof, relates to a controlled release of substances from source material, and, more particularly, but not exclusively, to release of substances which takes into account conditions of the source material tracked over time.

The present invention, in some embodiments thereof, also relates to selective delivery of pharmacologically effective doses and placebo doses of substances released from an inhalation device.

A broad aspect of some embodiments relates to release of substance(s) from a source material (e.g. a dose unit) which takes into account one or more effects associated with conditions in which the source material was contained or otherwise stored over time. In some embodiments, substances are released from a source material using an inhaler device, through which the substances are then pulmonary delivered to a user.

An aspect of some embodiments relates to release of precise amounts of at least one substance from a source material, e.g. by vaporization, by applying heating and/or airflow profiles selected, at least in part, according to conditions in which the source material was stored, optionally over time (e.g. weeks, months, years).

In some embodiments, airflow and/or heating profiles are selected to compensate for changes in substance composition which may have occurred in the source material, and were optionally effected by the storage conditions and/or the storage duration. In some embodiments, when the source material comprises a botanical substance, e.g. cannabis, natural degradation may change the substance composition over time, and may be accelerated (or instead slowed) as a result of the storage conditions.

In some embodiments, conditions such as temperature, humidity, exposure to light, a level of rattling, and/or other conditions are sensed and optionally recorded over time. In some embodiments, the source material is stored in cartridge which includes one or more sensors configured for measuring conditions inside and/or outside of the cartridge. In some embodiments, conditions measured over time are recorded at a logger or other memory component, optionally configured as part of the cartridge. In some embodiments, the cartridge is a small, compact hand-held cartridge, suitable for use with a personal inhaler device.

In some embodiments, the conditions are tracked throughout a lifecycle of the cartridge, for example from the manufacturing of the cartridge (such as from sealing of the source material inside the cartridge), through storage of the cartridge, transfer of the cartridge, distribution of the cartridge and optionally further during personal storing and/or using of the cartridge by an individual user.

In an example, conditions are tracked for a cartridge starting at manufacturing and ending, for example, at removal of the cartridge from the inhaler device at the end of use of the cartridge. A potential advantage of tracking conditions for a source material cartridge up to actual use of the cartridge in a personal inhaler device may include the ability to take into account conditions in which the cartridge was stored or placed also when the cartridge is with an individual user, and up to a point of actual use.

In some embodiments, in use, the inhaler circuitry reads and/or receives the condition data, and automatically selects (optionally, by calculating) parameters of heating (e.g. a target temperature range, a duration of heating) and/or parameters of airflow (e.g. a rate of airflow through the source material) based on the received condition data. In some embodiments, the selection is made based on known or estimated changes in composition for a specific source material which were measured in prior experiments or are otherwise known. In some embodiments, the selection of heating and/or airflow profiles is made at each use of the inhaler, for example, at each inhalation of the user from the inhaler device.

Some potential advantages of applying heating and/or airflow profiles which “compensate” for changes in composition of the source material that are affected by storage conditions, to then enable release of a predetermined amount of at least one substance from the source material, may include: simplifying supply chain aspects, for example by reducing a need to store the material in refrigeration conditions, allowing for preparing the source material (e.g., grinding) optionally long before packaging the source material in the cartridge; potentially lengthening a shelf life of the source material; potentially allowing an individual user more flexibility when storing the cartridge (such as in where to store, or for how long) before and/or during use in the inhaler; potentially supporting safety of usage in various environments and/or under variable or varying conditions, potentially lengthening the usage period per cartridge (such as the storage period and/or the usage period, e.g. starting when a cartridge is unsealed or placed into first use in an inhaler).

Another aspect relates to providing both a pharmacologically effective dose and a placebo dose from the same inhalation device, and optionally from the same source material.

When referring to the same source material it should mean similar species of source material. In some embodiments, same botanical material. In some embodiments, same botanical material extract. In some embodiments, same synthetic source material. In some embodiments, the same source material shall relate to doses vaporized from the same source material bulk. In some embodiments, same source material shall mean similar dose units of source material, in some embodiments similar dose units that are contained in a container or in a cartridge to be used with a meter dose inhaler device. In some embodiments, the same source material means source material doses that consist of a similar amount of source material, in some embodiments of the same source material species. In some embodiments the same source material means similar dose units of ground cannabis. In some embodiments similar weight of ground cannabis. In some embodiments the same source material means similar dose units comprising similar weighted amount of ground cannabis taken from the same cannabis inflorescence batch.

In some embodiments, it is desired to provide a user with a combination of doses where some of the doses would have a pharmacologically induced effect on the user, for example, include at least a certain amount of an active substance (e.g. a physiologically active substance and/or psychoactive active substance); and some of the doses would serve as a placebo (i.e. a placebo dose), whereby the placebo dose may bring about a clinical effect (e.g. reduce pain and/or nausea), even though it is not pharmacologically induced. In the placebo dose, the user would sense the provided dose as an actual (effective) dose, but would actually be provided with a dose and/or substance that only taste and/or scent similar to that provided in a pharmacologically effective dose. The placebo may bring about an effect which is induced psychologically and not pharmacologically.

In some embodiments, both the pharmacological effective doses and the placebo doses are released from the same inhalation device, and optionally from the same source material, but differ from each other in the type and/or amount of substances released. For example, when the source material comprises cannabis, a pharmacologically effective dose may include at least a certain amount of THC and/or other active cannabinoids; while a placebo dose may include just sensory substances such as terpenes, the vaporization of which contributes a typical scent and/or taste of vaporized cannabis, hence associated with the active cannabinoids. In some embodiments a placebo dose includes the release of a vapor.

In some embodiments, inhaler device circuitry (such as a controller of the device) is preprogrammed with heating and/or airflow profiles suitable to release selected substance(s) at selected amount(s) according to a delivery regimen which includes both pharmacologically effective and placebo doses. In an example, a heating profile for releasing certain sensory substances while avoiding or reducing the delivery of active substances may be set to reach a low enough temperature range in which only (or mostly) the sensory substances are released from the source material, while the active substances do not reach their vaporization point. In some embodiments a heating profile is designed to reach a target temperature in which at least one selected active substance does not reach its vaporization point, while at least one sensory substance is released, such as by reaching the sensory substance's vaporization point.

In some embodiments, the inhaler device circuitry (such as a controller of the device) is preprogrammed (or is preprogrammed to permit) for a placebo dose with heating to a temperature of less than 130° C., less than 120° C., less than 110° C., less than 100° C., less than 90° C., less than 80° C., less than 70° C., less than 60° C., less than 50° C., less than 40° C., less than 30° C.

In some embodiments, the inhaler device circuitry (such as a controller of the device) is preprogrammed (or is preprogrammed to permit) for a placebo dose with no heating, and having an airflow profile suitable to release selected substance(s) at selected amount(s) according to a delivery regimen. In some embodiments, the airflow profile includes controlling airflow valves to allow air to be drawn through the source material at interchangeable flowrates.

Another aspect relates to a method of delivering via inhalation at least one placebo dose from a THC-carrying material. The method comprises vaporizing at least one component of the THC-carrying material, wherein the at least one component is not THC; wherein the THC-carrying material is at a temperature below 130° C.

The THC-carrying material may be a botanical material, including plant material, including but not limited to Cannabis sativa, Cannabis indica, and Cannabis ruderalis. Other botanical materials are described herein below.

Other contemplated THC-carrying materials include purified preparations of THC (e.g. THC oil). Further examples of THC-carrying materials are provided herein below.

The THC-carrying material may also comprise additional components such as terpenes and/or other cannabinoids, which are further described below.

In some embodiments, the THC carrying material is maintained at a temperature of less than 130° C., less than 120° C., less than 110° C., less than 100° C., less than 90° C., less than 80° C., less than 70° C., less than 60° C., less than 50° C., less than 40° C., less than 30° C.

In some embodiments the delivery of the placebo dose requires no heating.

Another aspect relates to a method of delivering via inhalation one or more substances released from a source material (e.g. a terpene), the one or more substances having a selected sensory profile which is experienced by an inhaling user, the method comprising:
controlling heating of the source material to release the one or more substances from the source material, wherein the source material is maintained at a temperature less than 130° C. (e.g. less than 120° C., less than 110° C., less than 100° C., less than 90° C., less than 80° C., less than 70° C., less than 60° C., less than 50° C., less than 40° C., less than 30° C.); and
delivering the released one or more substances to the inhaling user such that the user experiences the selected sensory profile.

In some embodiments the sensory profile comprises patterns of airflow. In some embodiments patterns of interchangeable flowrates may provide some sensory aspects of the sensory profile. In some embodiments airflow patterns are applied in order to release by vaporization or convection some selected substances.

Exemplary source materials are described herein below.

In some embodiments, the delivery regimen is planned taking into account one or more of: physician instructions or recommendations, preferences of the specific user, preferences of users having similar characteristics, and the like. In some embodiments, the user is “blind” to the type of dose being delivered, so that the placebo doses is brought about psychologically (and not pharmacologically). In some embodiments of such uses, a physician who administers the dose to the user is “blind” to the type of dose being delivered, so that the placebo doses may serve as a “double blind” placebo.

In some embodiments, the release of certain substances while potentially avoiding or reducing the amount of others is performed at least partially based on the changes in source material composition, such as changes occurring over time and/or in changes associated with the conditions in which the source material was kept. In some embodiments, for example as described herein, environmental and/or storage conditions of the source material are sensed and optionally recorded; then, based on the recorded data, the heating and/or airflow profiles are selected or calculated.

An aspect of some embodiments relates to release of substance(s) from a source material such that a desired sensory profile is obtained. In an example, pharmacologically effective amounts of substances are reduced to a minimum or avoided, and substance(s) which are released and delivered are ones that allow a user to sense (e.g. taste, smell, appearance) a similar or close sensation as compared to when the pharmacologically effective substance is released and delivered, thereby acting as placebo.

In some embodiments, possible effects that an active substance (e.g. THC) may have on user perception are taken into account when deciding on the type and/or amounts of substances released. For example, THC alters the taste (such as impairs it or instead enhances it), and that effect is taken into account when delivering the placebo. Optionally, one or more types of terpenes (and their amounts) are released to achieve the desired sensory profile.

In some embodiments, release of selected substance(s) is controlled (such as automatically, e.g. by an inhaler controller) to obtain the desired sensory profile. In some embodiments, a heating profile of the source material is controlled to release the desired substance(s) at desired amount(s). In other embodiments, an airflow profile is controlled to release the desired substance(s) at desired amount(s). When the user is delivered the released substance(s) (via inhalation), they may experience the selected sensory profile.

In some embodiments, the user is not aware that a placebo is being delivered. Alternatively, the user knowingly receives (optionally, due to personal preferences) a dose that has a selected sensory profile which optionally does not include a clinically active substance.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

### Tracking Conditions of a Source Material Cartridge and Selecting, Based on the Tracked Conditions, Heating and/or Airflow Profiles for Release of One or More Substance(s) from the Source Material

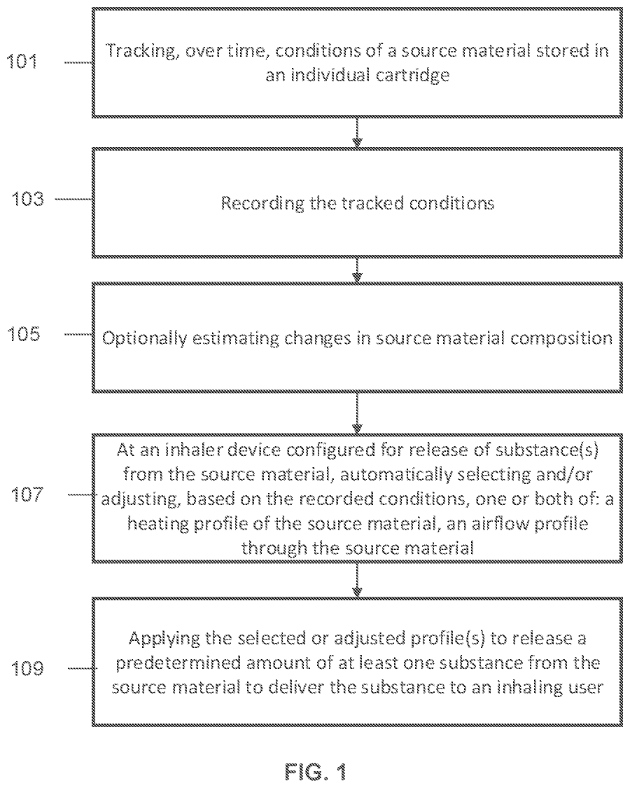

Referring now to the drawings, FIG. 1 is a flowchart of a method for tracking conditions of a source material stored in an individual cartridge over time, and controlling the release of at least one active substance from the source material in accordance with the tracked conditions, according to some embodiments.

In some embodiments, it is desired to track conditions such as environmental conditions of a source material from which one or more substances are eventually released to be delivered to an inhaling user. In some embodiments, the source material comprises a botanical substance in which natural and/or condition-affected degradation of certain substances may occur. In some cases, a chemical composition of substances in the source material changes over time, with the change potentially being affected by one or more conditions in which the source material is found, such as, but not limited to: temperature, humidity, exposure to light, a degree of rattling, physical pressure applied onto the source material, a type of sealing (or other wrap or packaging used), a type and/or structure of substrate material used (if at all), or others.

In some embodiments, conditions of a source material stored in an individual cartridge are tracked over time (101). In some embodiments, the conditions are tracked using one or more sensors, such as temperature sensors, humidity sensors, light sensors, pressure sensors. In some embodiments, timing is tracked for an individual cartridge, for example using a clock, timer, counter and/or other suitable means for recording and/or tracking time.

In some embodiments, conditions are tracked for source material contained within an individual cartridge, for example a cartridge of small dimensions which is intended for use with (e.g. inside) a personal hand-held inhaler device. In some embodiments, the cartridge contains a plurality of units (also referred to as “vaporchips” in the experiment described below) packed within an external casing or housing (for example, a single cartridge comprises between 5-10 units, 10-70 units, 50-200 units, 100-500 units or intermediate, larger or smaller number of units). In some embodiments, each unit comprises source material at a predefined amount.

In some embodiments, the sensor(s) that track conditions associated with storage of the cartridge are located inside the external casing or housing of the cartridge. Additionally or alternatively, one or more sensors are located outside the external casing or housing, for example, mounted externally on a wall of the housing. In some embodiments, condition(s) are tracked both externally to the cartridge housing and internally to the cartridge housing.

In some embodiments, the tracked conditions are recorded (103). Optionally, the tracked conditions are recorded over time, for example over 1 week, 1 month, 1 year, 2 years, 5 years, or intermediate, longer or shorter time periods. In some embodiments, conditions are tracked and recorded from initial manufacturing of the cartridge, for example at a lab or a factory. In some embodiments, conditions are tracked and recorded during the supply chain of the cartridge, for example during storage, during shipping, during transfer or other travel, during storage at the store (e.g. pharmacy). In some embodiments, conditions are tracked and recorded during personal use of the cartridge—for example when a user obtains the cartridge; stores the cartridge; uses the cartridge with their inhaler device; disposes of the cartridge or saves it for refilling; and/or other. In some cases, it may be potentially advantageous to track and record conditions of the cartridge both during “mass” travel and storage (such as when the cartridge is stored with additional, multiple cartridges) and during personal storage and/or use, when a specific cartridge is with the user.

In an example, environmental conditions such as temperature and humidity are tracked and recorded from the manufacturing of a cartridge, throughout massive storage, travel, and distribution; and then throughout personal storage, usage in an inhaler device, disposal or return (e.g. to a pharmacy or distribution center).

In some embodiments, the conditions tracked by the one or more sensor(s) are recorded at a memory component. In some embodiments, the memory component is internal to the cartridge and/or mounted on the cartridge, for example in the form of a logger, a memory chip, a flash memory, a volatile memory, a non-volatile memory, a cache memory, or any other suitable memory unit or storage unit. In some embodiments the memory is embedded in a processor or the like. Additionally or alternatively, the memory component is external to the cartridge, for example, the condition data obtained by the sensor(s) is communicated and recorded at external storage such as a system server, cloud-based storage, cloud-based computing services or others.

In some embodiments, optionally, the recorded conditions are analyzed to estimate changes in the source material composition (105). In some embodiments, one or more algorithms are applied to the collected data to deduce changes in a chemical composition of the source material and/or to deduce a current chemical composition of the source material. In some embodiments, the applied algorithms tie between known (and/or expected) effects of the storage conditions tracked for a cartridge, and the state (e.g., current chemical composition) of the source material.

In some embodiments, the analysis is performed based on input including one or more of: data regarding the plant strain being used, a duration of storage, the logged environmental conditions, such as temperature, humidity and exposure to light.

In some embodiments the analysis is performed during the operation of an inhalation device operative to deliver a substance to an inhaling user by heating the source material. In some embodiments, the analysis is performed based on input including environmental conditions measured during the operation of the device, such as the environment temperature, humidity, and barometric pressure.

In some embodiments the analysis is performed based on input including other parameters measured during the operation of the device, such as the temperature of a heating element, which is configured to heat the source material to extract the substance; a measured voltage and/or electrical resistance, e.g. of a resistive heating element, a measured air flow rate, such as air flow rate passing through the source material.

In some embodiments the analysis is performed based on input including a combination of one or more of the storage conditions, the storage duration, environmental conditions during the operation of the device and other parameters measured during the operation of the device.

In some embodiments, output data provided by the analysis may include an operation profile, including one or more of: a heating profile for the source material (for example a heating duration, a target temperature, a target voltage/current to be applied to a heating element of the source material); an airflow profile (for example a volume and rate of airflow, optionally by controlled operation of airflow valves); an amount of source material to be delivered (for example delivering one or more substances released from a selected mass or volume of the source material, for example in embodiments in which the source material contains bulk source material. In some embodiments, delivery of one or more substances from a selected portion of the source material is done by heating a selected portion of the source material, optionally by heating a selected area of a flat dose unit); and/or other output data.

In some embodiments, algorithms applied for analyzing the data are updated (optionally over time) based on experimental results, based on results of other users, based on literature data, and/or other sources. Such updating may take place, for example, via a cell phone application used with the inhaler device, via communication of the inhaler device itself, and/or other data transfer or communication means.

In some embodiments, the analysis calculates or estimates degradation of substances in the source material. For example, when the source material comprises cannabis, decarboxylation of THCA into THC is assessed or estimated, such as based on the duration of storage and/or the storage conditions (e.g. temperature, humidity). In some embodiments, based on the analysis, operation profile parameters are calculated and/or otherwise selected based on the tracked conditions and/or a based on a calculated or estimated degradation of substances in the source material.

In some embodiments, the analysis includes processing of data collected over time, such as by applying of machine learning algorithms. In some embodiments, patterns or trends in the collected data (e.g. in measured temperatures, measured humidity levels etc.) are identified, and are optionally compared to known and/or previously measured data.

In some embodiments, data collected over time is used to train and/or develop a machine learning algorithm. In some embodiments, regression models are used for the calculation. In some embodiments, the regression models include linear regression models. In some embodiments the calculation takes into account one or more of: calculated or estimated changes in the composition of the source material, the plant strain being used, a duration of storage, the logged environmental conditions, such as temperature, humidity and exposure to light. In some embodiments the calculation takes into account environmental conditions measured during the operation of the device, such as the environment temperature, humidity, and barometric pressure. In some embodiments the calculation takes into account other parameters measured during the operation of the device, such as a temperature of a heating element, which is configured to heat the source material to extract the substance and/or a measured or calculated temperature of the source material during the heating; a measured voltage and/or electrical resistance, a measured air flow rate. In some embodiments the calculation takes into account a combination of one or more of the storage conditions, the storage duration, environmental conditions during the operation of the device and other measured parameters during the operation of the device.

In some embodiments, the source material cartridge is placed into use with an inhaler device. In some embodiments, when used with the inhaler device, each unit of a cartridge (such as described hereinabove) is moved into a use-position in which the source material is heated and airflow is passed through, to release one or more substances from the source material (e.g. via vaporization, aerosolization, convection).

In some embodiments, one or both of a heating profile of the source material and an airflow profile through the source material are automatically selected and/or adjusted, at least partially based on the recorded conditions, to release selected amount(s) and/or type(s) of substances from the source material (107).

In some embodiments, the heating and/or airflow profiles are selected at a controller of the inhaler device which reads and/or receives data associated with the recorded conditions from a logger or other memory component, for the specific cartridge being used. In some embodiments, reference is made to a database or a lookup table tying between cartridge storage conditions and parameters of heating and/or airflow and/or amount of source material to be used which are suitable for releasing of certain substance(s) at certain amount(s) from the source material. In some embodiments, heating and/or airflow parameters are calculated, for example based on known experimental data tying between storage conditions of the source material (e.g. temperature, humidity, exposure to light, duration of storage) and the expected and/or measured composition of substances in the source material at one or more times, under the specific storage conditions.

In some embodiments, selection of heating and/or airflow parameters is performed at each activation of the inhaler device and/or at each delivery to a user, for example so that the most updated data regarding storage conditions and/or storage duration is read from the logger (or other memory component) and processed immediately before release of the substance(s) from the source material to the inhaling user.

In some embodiments, selection of a heating profile involves selection of one or more of: a target temperature for the source material, a duration of heating, a level of electrical power supplied to a heating element of the source material, a heating rate, and heating modulations and/or patterns.

In some embodiments, selection of an airflow profile involves selection of one or more of: a rate of airflow passing through the source material, a volume of airflow, a duration of passing of airflow through the source material, and modulations and/or patterns.

In some embodiments, the airflow and/or heating parameters are selected to compensate for estimated and/or measured changes that occurred in the source material as a result of the storage duration and/or storage conditions in which the cartridge was maintained. Optionally, parameters are selected so that a released dose of a substance from the source material remains stable regardless of the storage time and/or storage conditions. Compensating for variability in the source material which is a result of storage time and/or storage conditions by adjusting the heating and/or airflow profiles may be potentially advantageous in that precise doses may be repetitively released from source material that had been stored in variable conditions and/or over various different periods of time.

In an example, a similar dose of an active substance may be released from both source material that had been stored in refrigeration over time and from source material that had been stored in room temperature over time, by modifying the parameters of heating (for example—the target temperature to which the source material is heated, the duration of heating).

In some embodiments, airflow parameters and heating parameters are selected in consideration of each other due to that airflow may affect the results of heating, or vice versa.

In some embodiments, the selected and/or adjusted heating and/or airflow profiles are applied to release a predetermined amount of at least one substance from the source material, and to deliver that substance to an inhaling user (109).

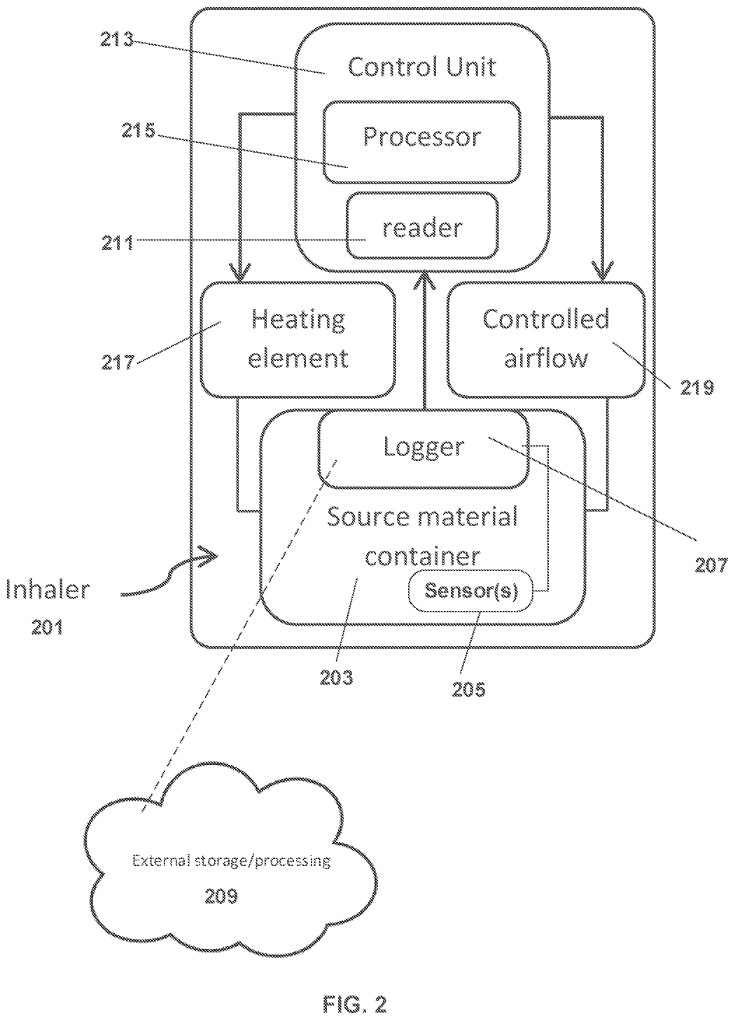

FIG. 2 is a schematic diagram of an inhaler system for controlled release of at least one substance from a source material, the system configured for sensing and/or recording conditions of a source material, according to some embodiments.

In some embodiments, an inhaler device 201 is a hand-held device suitable for personal use. In some embodiments, the inhaler device comprises and/or is configured to receive therein and/or operably attach to a source material container or cartridge 203 in which source material is contained.

In some embodiments, the source material cartridge comprises a housing containing the source material. Optionally, the source material is contained within separate units inside the cartridge, which can be independently placed in a use position in the inhaler device. In an example, the cartridge is disc shaped and comprises a carousel of multiple units, each unit including source material at a selected amount. In another example, the cartridge is elongate (e.g. rectangular) and comprises, for example, stacked units.

In some embodiments, the cartridge is shaped and size to fit inside or otherwise attach to the inhaler device, for example having a volume of less than 200, 100, 70, 50, 40, 30, 20, 15 cm\hat{ }3, or intermediate, larger or smaller volume and a weight of less than 100, 80, 60, 50, 40, 30, 20, 15, and 5 grams or intermediate, larger or smaller weight. In some embodiments, the cartridge is small enough to be held and moved by a user's hand, for example during placing of the cartridge into use with the inhaler and/or when removing an empty cartridge from the inhaler.

In some embodiments, the source material is in the form of solid, ground particles. In some embodiments, the source material is in the form of powder. In some embodiments, the source material is in the form of liquid, for example, oil.

In some embodiments, the source material contains a botanical substance which has maintained its natural granular form. For example, cannabis in which trichome integrity has been maintained.

In some embodiments, the source material comprises a purified substance, for example purified CBD, THC, other cannabinoids and/or terpenes. In some embodiments, the source material comprises an isolated substance. In some embodiments, the source material comprises a synthetic substance.

According to some embodiments, the source material from which the at least one substance is released comprises botanical material, including, for example: Cannabis sativa, Cannabis indica, Cannabis ruderalis, Acacia spp, Amanita muscaria, Yage, Atropa belladonna, Areca catechu, Brugmansia spp., Brunfelsia latifolia, Desmanthus illinoensis, Banisteriopsis caapi, Trichocereus spp., Theobroma cacao, Capsicum spp., Cestrum spp., Erythroxylum coca, Solenostemon scutellarioides, Arundo donax, Coffea arabica, Datura spp., Desfontainia spp., Diplopterys cabrerana, Ephedra sinica, Claviceps purpurea, Paullinia cupana, Argyreia nervosa, Hyoscyamus niger, Tabernanthe iboga, Lagochilus inebriens, Justicia pectoralis, Sceletium tortuosum, Piper methysticum, Catha edulis, Mitragyna speciosa, Leonotis leonurus, Nymphaea spp., Nelumbo spp., Sophora secundiflora, Mucuna pruriens, Mandragora officinarum, Mimosa tenuiflora, Ipomoea violacea, Psilocybe spp., Panaeolus spp., Myristica fragrans, Turbina corymbosa, Passiflora incarnata, Lophophora williamsii, Phalaris spp., Duboisia hopwoodii, Papaver somniferum, Psychotria viridis, spp., Salvia divinorum, Combretum quadrangulare, Trichocereus pachanoi, Heimia salicifolia, Stipa robusta, Solandra spp., Hypericum perforatum, Peganum harmala, Tabernaemontana spp, Camellia sinensis, Nicotiana tabacum, rusticum, Virola theidora, Voacanga africana, Lactuca virosa, Artemisia absinthium, Ilex paraguariensis, Anadenanthera spp., Corynanthe yohimbe, Calea zacatechichi, Coffea spp. (Rubiaceae), a Sapindaceae, Camellia spp., Malvaceae spp., Aquifoliaceae spp., Hoodia, spp. Chamomilla recutita, Passiflora incarnate, Camellia sinensis, Mentha piperita, Mentha spicata, Rubus idaeus, Eucalyptus globulus, Lavandula officinalis, Thymus vulgaris, Melissa officinalis, Aloe Vera, Angelica, Anise, Ayahuasca (Banisteriopsis caapi), Barberry, Black Horehound, Blue Lotus, Burdock, Camomille/Chamomile, Caraway, Cat's Claw, Clove, Comfrey, Corn Silk, Couch Grass, Damiana, Damiana, Dandelion, Ephedra, Eucalyptus, Evening Primrose, Fennel, Feverfew, Fringe Tree, Garlic, Ginger, Ginkgo, Ginseng, Goldenrod, Goldenseal, Gotu Kola, Green Tea, Guarana, Hawthorn, Hops, Horsetail, Hyssop, Kola Nut, Kratom, Lavender, Lemon Balm, Licorice, Lion's Tail (Wild Dagga), Maca Root, Marshmallow, Meadowsweet, Milk Thistle, Motherwort, Passion Flower, Passionflower, Peppermint, Prickly Poppy, Purslane, Raspberry Leaf, Red Poppy, Sage, Saw Palmetto, Sida cordifolia, Sinicuichi (Mayan Sun Opener), Spearmint, Sweet Flag, Syrian Rue (Peganum harmala), Thyme, Turmeric, Valerian, Wild Yam, Wormwood, Yarrow, Yerba Mate, Yohimbe, Galanthus woronowii, Rauvolfia reserpine, Ginkgo biloba, Boswellia papyrifera, Centella asiatica, Rosmarinus officinalis, Eschscholtzia californica, Abutilon indicum, Bacopa Monnieri, Rhodiola rosea, Huperzia serrata, Erythrina mulungu, Hippeastrum vittatum, Afzelia africana, Crocus sativus, Punica granatum, Physostigma venosum, Geissospermum vellosii, Salsola oppositefolia, Mexican peyote cactus, Gastrodia elata, Narcissus spp., Magnoliaceae spp., and any part and any combination thereof.

According to some embodiments, the botanical material includes plant material, including for example Cannabis sativa, Cannabis indica, and Cannabis ruderalis.

According to some embodiments, the substance includes a cannabinoid or an acidic form of a cannabinoid. Examples include Δ9-tetrahydrocannabinol (THC), cannabidiol (CBD), cannabigerols (CBG), cannabichromenes (CBC), cannabinol (CBN), cannabinodiol (CBDL), cannabicyclol (CBL), cannabielsoin (CBE), cannabidivarin (CBDV), tetrahydrocannabivarin (THCV) and cannabitriol (CBT) and acidic forms of the aforementioned.

According to some embodiments, the substance includes Δ9-tetrahydrocannabinol (THC), tetrahydrocannabinolic acid (THCA), cannabidiol (CBD) and/or cannabidiolic acid (CBDA).

According to some embodiments, the substance comprises legally approved non-medical drugs, such as caffeine, cathinone, cathin, nicotine, Myristicin (Methoxysafrole), trans-neoclerodane diterpenoids, such as salvinorin A, sesquiterpene lactones, such as germacranolides, dextromethorphan, 4-ACO-DMT (also known as O-Acetylpsilocin or psilacetin), kavalactones (or kavapyrones), such as kavain, dihydrokavain, methysticin, dihydromethysticin, yangonin, and desmethoxyyangonin; substances present in Turnera diffusa, substances present in Argyreia nervosa, such as ergot alkaloids; substances present in Echinopsis pachanoi and/or in Lophophora williamsii, such as mescaline, 3,4-dimethoxyphenethylamine, 3-Methoxytyramine, 4-hydroxy-3-methoxyphenethylamine, 4-hydroxy-3,5-dimethoxyphenethylamine, anhalonidine, anhalinine, hordenine, and tyramine; aporphine; substances present in Amanita muscaria, such as muscarine, ibotenic acid, muscimol and Muscazone; hallucinogenic tryptamines present in Incilius alvariusvenom venom, such as 5-MeO-DMT and Bufotenin.

According to some embodiments, the substance is or includes a terpenoid, alkaloid or cannabinoid. For example, in some embodiments, the substance is a diterpenoid such as, but not limited to salvinorin A from salvia. In other embodiments, the substance is an alkaloid such as, but not limited to, benzoylmethylecgonine from the coca plant, or the substance is a tryptamine such as psylocibin from mushrooms. In alternative embodiments the substance is dimethyltryptamine (DMT) from a variety of botanicals. In further embodiments, the substance is nicotine from tobacco. In further embodiments, the substance is a terpenoid, e.g., limonene, a-pinene, β-myrcene, linalool, β-caryophyllene, caryophyllene, nerolidol or phytol, present in various botanical (e.g. plant) forms.

Substances (from an organic source or from a non-organic source) which have medicinal properties and are suitable for use with delivery systems and/or devices and/or methods for example as described herein are also to be contemplated by this application.

In some embodiments, the cartridge comprises one or more sensors 205 configured for measuring conditions inside and/or outside of the cartridge, for example, temperature sensor(s), light detection sensor(s), humidity sensor(s), pressure sensor(s).

In some embodiments, measurement by the sensors is performed continuously. In some embodiments, measurement is performed at predefined time intervals, for example, once a day, once an hour, once every 10 minutes, once every 5 minutes, once a minute or intermediate, longer or shorter time intervals. In some embodiments, the measurement begins prior to manufacturing of the cartridge and is optionally perfomed during the process of production of the source material. In some embodiments, the measurement begins at manufacturing of the cartridge, for example once a cartridge is sealed. In some embodiments, the measurement continues until commencement of distribution. In some embodiments the measurement is continued during the storage period and/or during distribution and/or during the usage period.

Alternatively, the measurement is stopped when the cartridge is placed into use with the inhaler device, and/or when the cartridge is unsealed (e.g. by the user) from its packaging (optionally, removed from a vacuum package).

In some embodiments, the measurement scheme (e.g., when to start and stop tracking, which conditions are tracked, the measurement rate and/or other parameters of measurement) are determined in accordance with one or more of: the type of source material, expected shelf-life of the source material, the cartridge materials and/or structure, the type of sealing used (e.g., how isolated the source material is from its environment), an expected or estimated storage time until use in the inhaler.

In some embodiments, the measurement scheme is defined according to parameters such as shelf life which were tested in a lab and/or at a manufacturing facility.

In an example, the measurement is performed over a total time period of up to, for example, 1 months, 5 months, 16 months, 18 months, 24 months, 36 months or intermediate, longer or shorter time periods, for example starting from manufacturing of the cartridge (e.g. from the point of vacuum sealing the cartridge at a lab or factory).

In some embodiments, data measured by the one or more sensors is recorded at a logger 207. Optionally, the logger is contained inside the cartridge and/or is mounted onto a housing of the cartridge. In some embodiments, data recorded at the logger is communicated to an external storage or processing means 209 (e.g. an external server, cloud memory, cellular phone storage, and the like).

In some embodiments, when the cartridge is in use with the inhaler device, condition measurement data from the logger is read by and/or transferred to a reader 211 of the inhaler device control unit 213. In some embodiments, a processor 215 (a controller) at the control unit is configured to select and/or calculate, based on the condition measurement data received from the logger, parameters of heating and/or airflow to be applied.

In some embodiments, the control unit controls a heating element 217 which heats the source material according to the selected heating parameters. In some embodiments, the control unit controls airflow 219 through the source material (and/or otherwise through the inhaler device), for example by controlling one or more valves (not shown), according to the selected airflow parameters.

In some embodiments, the heating parameters and the airflow parameters are selected to release a predetermined amount of one or more substances from the source material, and deliver those substances to a user inhaling from the device (e.g. through a mouthpiece, not shown).

A potential advantage of setting heating and/or airflow parameters according to conditions tracked for a specific cartridge may include the ability to release stable, accurate doses of a substance even after long periods of storage (e.g., months or years), during which the source material may have degraded or otherwise changed. In an example, for releasing THC from cannabis, decarboxylation (of THCA into THC) over time is taken into account and the heating and/or airflow parameters can be set accordingly, to “compensate” for the change in composition.

In some embodiments, unwrapping of a cartridge from its package and/or a first use of the cartridge in the inhaler device (in which the source material may be unsealed) may affect changes in composition (e.g. degradation) of the source material, and are taken into account when selecting or calculating the heating and/or airflow parameters.

In some embodiments, the control unit 213 is in communication with a cellular phone application through which data may be received from a user of the inhaler device and/or communicated to the user.

In some embodiments, the changes in heating and/or airflow are automated and the user is “blind” to the change in sense that they can continue inhaling from the device as they are used to and be delivered their predefined amounts of substance(s) released from the source material.

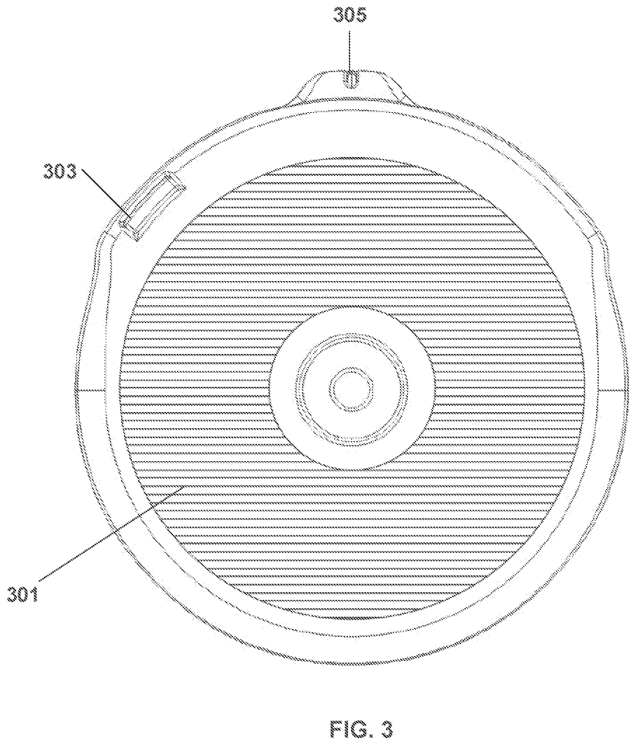

FIG. 3 is an example of a source material cartridge for use with an inhaler device, the source material cartridge comprising a logger for recording condition data, according to some embodiments.

In the example of FIG. 3, a rounded, disc shaped cartridge 301 shown. In some embodiments, the source material is stored inside the cartridge, for example divided into multiple independently usable units (not shown).

In some embodiments, when the cartridge is placed into use with an inhaler device, a unit of source material is transferred into a use-position within the inhaler, for example via a pathway 305 (e.g. a slot). In some embodiments, the unit is pulled or pushed from the cartridge housing and into the use position of the inhaler, for example via a mechanical arm or other transfer means.

In some embodiments, the cartridge comprises a logger 303 which records condition data for the cartridge, for example condition data measured by one or more sensors.

In some embodiments, the logger is embedded in an identification label of the cartridge, for example, an RFID label. In use, the RFID label is identified by the inhaler device, and data from the logger is transferred and/or otherwise read by inhaler device circuitry. In some embodiments the RFID label includes data regarding the cartridge, for example: the time when the cartridge was manufactured, the time that passed since the cartridge was manufactured, the time in which the cartridge was opened or firstly used, the number of source material units in the cartridge; the type(s) of source material; the types of substance(s); the amount of substance; which source material(s) are contained in each dose unit (if, for example, different units in the cartridge contain different source materials); the location of each dose unit within the cartridge (e.g. with respect to the cartridge housing (where, for example, a unit closer to the wall of the housing may be more prone to effects by environmental conditions) and/or with respect to other units); which dose units had been used; manufacturing date; expiration date; manufacturing batch information, use information and/or other cartridge related data.

In some embodiments the RFID is associated and/or configured to communicate with at least one sensor. In some embodiments, the inhaler comprises or is associated with an RFID reader/writer. In some embodiments, the RFID reader/writer identifies the cartridge upon loading of the cartridge into the inhaler. In some embodiments, the RFID reader/writer is programmed to write data to the tag of the cartridge, for example: the amount of source material units that have been used from the cartridge; the numbering and/or location of the source material units used (in an example, the units are serially numbered according to their respective location in the cartridge, but can be used in any order); the amount of substance that was extracted and/or the amount of substance remaining in each used unit; and/or other cartridge usage related data.

### Balancing Between Pharmacologically Effective Doses and Placebo Doses Delivered to a User Via Inhalation

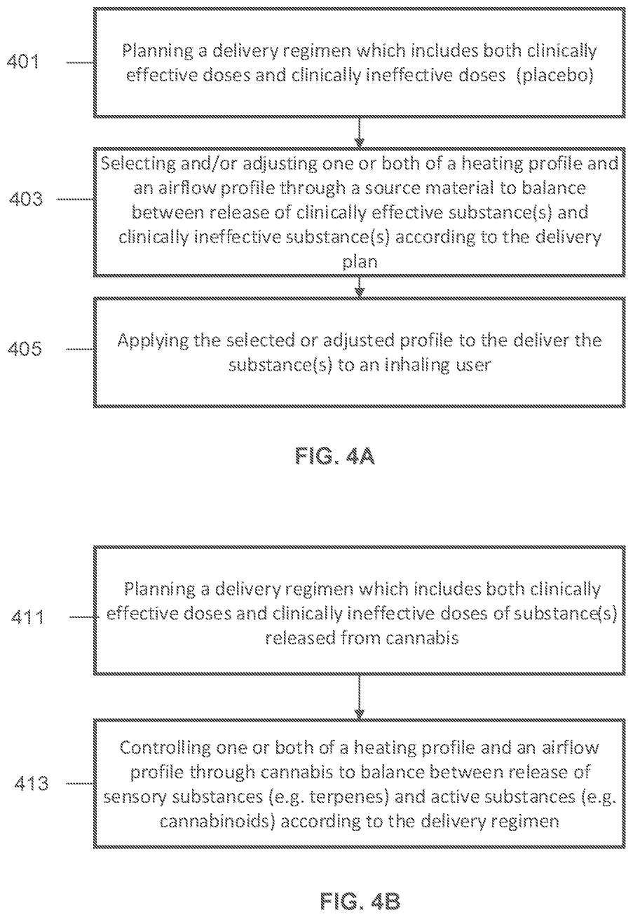

FIGS. 4A-B are flowcharts of methods for balancing release of pharmacologically effective substances or doses and placebo substances or doses (placebo doses) from a source material, according to some embodiments.

In some embodiments, it is desired to provide a user with a combination of substances and/or doses that would have a clinical effect on the user (e.g. alleviate symptoms like pain, nausea) and substances and/or doses that would be pharmacologically ineffective, providing only a sensory or placebo effect on the user.

In some embodiments, the treated symptoms are associated with conditions such as depression, Tourette, PTSD, anxiety, bowel disease, and/or others.

In some embodiments, a pharmacologically effective dose and/or substance is one that had proven, in previous deliveries to the same user and/or to other users, to have a clinical effect brought about by the active substance. For example, where a blood concentration level of the delivered substance is above a threshold; where a change in one or more physical measures is evident and quantifiable such as blood pressure, heart rate, body fever, pupil dilation, and/or other measures.

In some embodiments, stability of the dose of active substance released is maintained regardless (or despite) of the storage duration and/or storage conditions of the specific cartridge, potentially allowing to provide a user with repetitive, known doses which would be clinically effective for that user.

In some embodiments, a placebo dose and/or substance is one that the user would sense as an actual (effective) dose, without having a pharmacologically induced effect on the user. For example, the placebo dose or substance would provide the user only with a taste and/or scent similar to that of the effective dose or substance. In some embodiments, the placebo dose would provide the appearance and “feel” of a vapor being released. In some embodiments, the placebo dose or substance would not cause a major change in psychoactive level, for example, would not cause a user to be “high”, yet may still have a limited psychoactive effect on the user—for example, cause the user to feel calmer as if they were receiving an actual (pharmacologically effective) dose or substance.

In some embodiments, the placebo dose includes the delivery of only a small amount of an active substance, for example small enough so as not to physiologically affect the user, for example small enough so as not to be detected in the user's blood, or having a blood concentration level lower than a predetermined threshold. In some embodiments, the placebo dose includes the delivery of one or more substances that have a sensory effect on the user, for example have a taste and/or scent and/or appearance and/or thermal effect (e.g. a feeling of warm or hot vapor) and/or otherwise the “feel” of a vapor which may be identified by the user as an actual (effective) dose.

In some embodiments, only sensory substances are released from the source material and delivered to the user, while active substances are avoided. In an example, a placebo dose released from cannabis includes sensory substances such as terpenes, while delivery of active substances such as THC is avoided. Optionally, release of cannabinoids is avoided. In some embodiments a placebo dose released from cannabis includes vapor.

In some embodiments, as described for example in the flowchart of FIG. 4A, a delivery regimen which includes both pharmacologically effective doses and placebo doses is planned for a user (401). In some embodiments, the regimen is planned according to one or more of: user preferences, prescribed instructions (e.g. by a treating physician), local regulations, and/or others.

In some embodiments, for each of the scheduled doses, one or both of a heating profile and an airflow profile applied at an inhaler device for release of substances from a source material are selected according to the delivery plan (403). In some embodiments, parameters such as a target temperature to which the source material is heated are selected according to the type of dose.

For example, for a placebo dose, a target temperature range is selected so that only selected substances are released (e.g. only non-pharmacologically effective substances are released, while avoiding or reducing pharmacologically effective substance release) and/or to release only limited amounts (e.g. release only placebo amounts which are small enough so as not to actively affect the user). In the example of cannabis, the target temperature may be selected at a range in which terpenes are released by vaporization and/or convection, but THC release is limited or avoided.

In some embodiments, the selected airflow and/or heating profiles are then applied (such as by the device controller) to release and deliver the substance(s) to an inhaling user (405).

In some embodiments, the delivery regimen includes multiple doses in which some of the doses are pharmacologically effective, while others are not. In some embodiments, the user is not aware of the type of dose being delivered, so that the placebo dose may still have a placebo effect on the user. Delivery of placebo doses may be advantageous, for example, in clinical trials (e.g. for facilitating a double blind effect). Delivery of placebo doses may be advantageous, for example, during a titration/calibration stage, when a user starts a treatment by an active substance, and the amounts of active substance delivered are gradually increased to help the user become accustomed to the treatment. In such situation, placebo doses may be intervened between clinically effective doses to assist a user in becoming accustomed to the taste, scent, vapor and/or other sensation-related effect of the active substance. The placebo doses may be randomly introduced or according to a predetermined program.

FIG. 4B is a flowchart of a specific example for delivery of pharmacologically effective and placebo doses released from cannabis. In some embodiments, a delivery regimen including pharmacologically effective doses and placebo doses released from cannabis is planned (411). In some embodiments, the delivery plan is for therapeutic purposes, for example for alleviation of symptoms (e.g. pain). Additionally or alternatively, the delivery plan is targeted at recreational purposes, for example when a user intends to feel a psychoactive effect (e.g. “high”).

In some embodiments, a user inserts their preferences into the system (such as via a cell phone app in communication with the inhaler controller), and the plan is constructed accordingly. For example, a user inserts their scheduled activities and/or times in which they prefer to feel a psychoactive effect and/or times in which they prefer not to feel a psychoactive effect (e.g during working, driving) and/or times in which they prefer to feel alleviation of symptoms; and the delivery plan is generated accordingly, balancing between doses that would cause an actual physiological effect and/or psychoactive effect and doses that would only feel to the user as if the actual active substance (e.g. THC) is being delivered, for example, doses that would smell and/or taste and/or otherwise feel as if an active substance is being delivered.

In some embodiments, placebo doses may be delivered in situations in which, for example, the user is not authorized to receive an active substance (e.g. is under-aged); the user has been delivered a maximal amount allowed or selected (such as per a certain time period);

In some embodiments, one or both of a heating profile and/or an airflow profile through the cannabis are controlled to release various substances (and/or various amounts of substances) according to the delivery plan (413). Optionally, during delivery of placebo doses, only sensory substances such as terpenes are released from the cannabis and delivered to the user; and during delivery of pharmacologically effective doses, active substances such as cannabinoids (e.g. THC, CBD) are released from the cannabis and delivered to the user. Optionally, during delivery of placebo doses, release of active substances such as cannabinoids is avoided. Optionally, during delivery of placebo doses, release of THC is avoided, while at least one terpene is released. Optionally, during delivery of placebo doses, release of THC is avoided, while vapor, scent and/or taste of cannabis are provided, for example by release of terpenes.

### Tracking Conditions of a Source Material Cartridge and Controlling the Heating and/or Airflow Profiles for Selectively Delivering One or More Substances Released from the Source Material

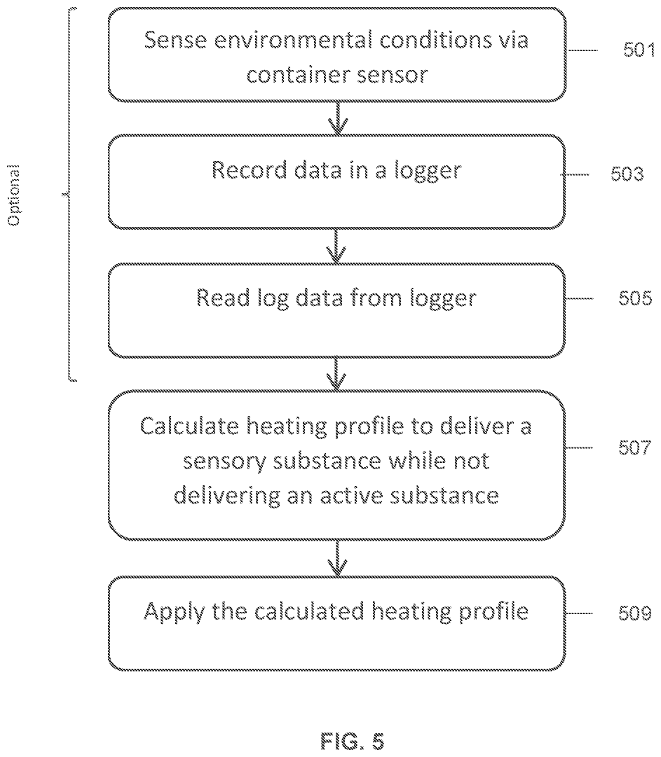

FIG. 5 is a flowchart of a method for sensing and recording conditions of a source material over time, and selecting a heating profile for the source material so as to selectively release placebo doses, according to some embodiments.

In some embodiments, for example as described hereinabove, environmental conditions for a source material stored in a container or cartridge are measured or sensed, e.g. via one or more sensors of the cartridge (501). The sensed conditions may include, for example, temperature (inside and/or outside the cartridge housing), humidity (inside and/or outside the cartridge housing), exposure to light, a level of rattling of the cartridge (e.g. during travel), a level of pressure applied onto the cartridge, and/or others.

In some embodiments, the sensed conditions are tracked over time and recorded at a logger (503).

In some embodiments, when the cartridge is placed for use with an inhaler device, condition data from the logger is read (e.g. by a controller of the inhaler device) (505). Optionally, the condition data is processed to determine changes in composition of the source material, for example as a result of degradation, as a result of the environmental conditions in which the cartridge was stored, or a combination thereof (i.e. when degradation is accelerated due to storage conditions, for example due to a high environmental temperature, due to major exposure to light, and the like).

In some embodiments, a heating profile to be applied to the source material is calculated, for example to deliver a placebo dose which includes sensory substances but does not include the delivery of an active substance to the user (507). (Additionally or alternatively, a heating profile may be calculated to deliver clinically effective doses which do include the delivery of one or more active substances to the user).

In some embodiments, the calculated heating profile defines parameters of heating such as a target temperature range, a maximal temperature which should not be surpassed, a frequency in which heating is applied, a duration of heating.

In some embodiments, the calculated heating profile is applied to heat the source material and release the selected substance(s) at selected amounts and deliver them to the inhaling user (509).

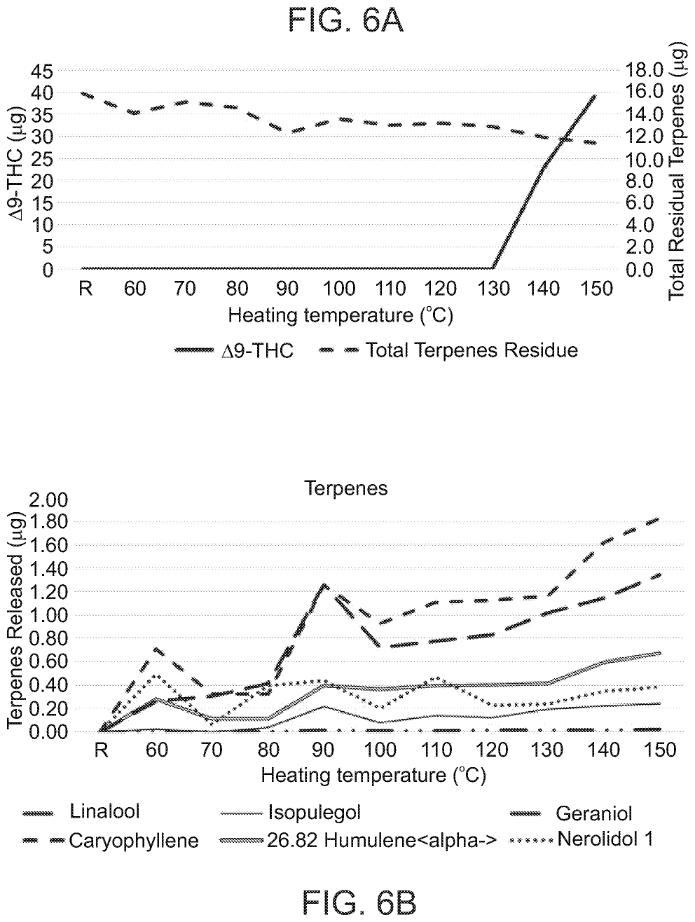

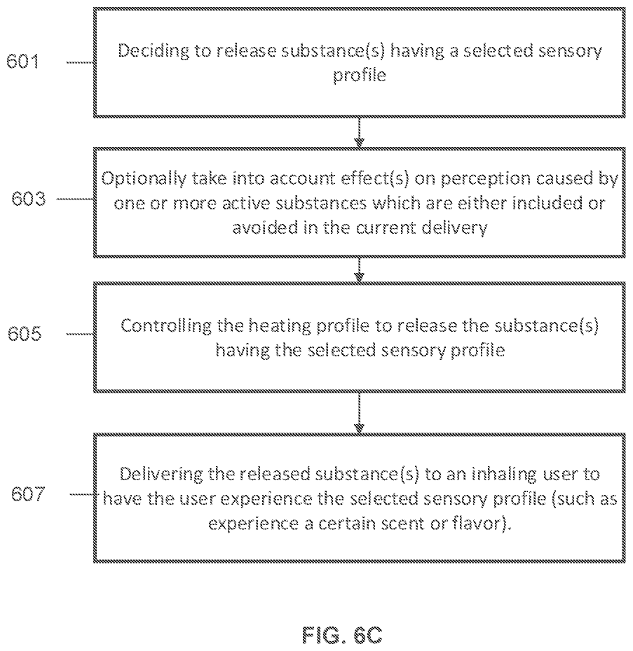

FIGS. 6A-B are vaporization plots showing an example of controlled release of THC and various terpenes from cannabis, according to some embodiments.

In some embodiments, release of selected substances from source material is controlled by setting a target temperature (or a target temperature range) to which the source material is heated. In some embodiments, the target temperature is defined according to the vaporization temperatures of different substances from the source material, including for example one or more substances which are desirably released, and/or one or more substances which are avoided or are reduced in amount when released.

The vaporization plots in FIGS. 6A-B show results of an experiment performed by the inventors in which samples of 135 mg of ground cannabis were heated to several target temperatures, including 60° C., 70° C., 80° C., 90° C., 100° C., 110° C., 120° C., 130° C., 140° C. and 150° C. At each of the target temperatures, vaporized THC was measured in the aerosol. At each of the target temperatures, the residual mass of ground cannabis was analyzed using Gas Chromatography-Mass Spectrometry (GC/MS) to determine existence of terpenes, such as: Linalool, Isopulegol, Geraniol, Caryophyllene, 26.82 Humulene<alpha-> and Nerolidol. The total terpenes residue (as remaining in the ground cannabis) is indicated by the dashed line in FIG. 6A. Based on the residual mass of terpenes, the released (vaporized) amount terpenes was calculated, by subtracting the measured residual mass from a respective reference mass. FIG. 6B shows, for the various terpenes released from the ground cannabis, the amount released as depending on the temperature to which the ground cannabis was heated.

As can be observed from FIG. 6A, THC (indicated by the continuous line) release started at a target temperature of 130 degrees C., and increased with the rising of the target temperature (up to 150 degrees C.). As can be observed from FIG. 6B, the estimate of released terpenes showed vaporization of terpenes from the cannabis at temperatures lower than 130 degrees C., such as starting from a temperature of 60 degrees C. or even lower.

Therefore, in some embodiments, by controlling a target heating temperature range, certain substances such as terpenes can be selectively released while other substances such as THC may be reduced or avoided. For example, by heating a unit comprising ground cannabis to a temperature between 60-130 degrees Celsius, selective release of terpenes may be achieved while release of THC may be minimized or even fully avoided.

FIG. 6C is a flowchart of a method for releasing substance(s) having a selected sensory profile (e.g. flavor, scent), according to some embodiments.

In some embodiments, it is desired to provide a user with a dose having a selected sensory profile (601), for example, which has a distinguishing scent and/or taste and/or appearance and/or “feel” of a vapor.

In some embodiments, the dose is a clinically effective dose, which includes one or more active substances, for example as described herein. Alternatively, in some embodiments, the dose is a placebo dose.

Optionally, when determining the desired sensory profile, one or more effects that an active substance may have on sensory perception are taken into account (603). The active substance(s) may be included in the dose, or instead avoided or reduced, but their effect on perception may still be considered. For example, THC delivered to a user may affect their sense of smell and/or taste, and therefore, in some embodiments, when deciding on the desired sensory profile, a potential effect of THC may be taken into account.

In some embodiments, the heating profile is controlled (for example automatically, such as by the inhaler device controller) to release the substance(s) having the selected sensory profile (605). In an example, terpenes are released from cannabis, while THC may be reduced or avoided.

In some embodiments, the dose including the selected released substance(s) is delivered to the user, who in turn experiences the sensory effects (e.g. taste, scent, vapor related sensations) induced by the released substance(s).

In some embodiments, conditions such as environmental conditions are tracked for the source material, optionally over time, and the heating profile is selected and/or adjusted based on the estimated and/or calculated changes in source material composition associated with the conditions. For example, an amount and/or type of terpenes released from cannabis are controlled based on their expected presence in the source material which may have been affected by the storage conditions and/or storage duration.

An additional experiment was performed by the inventors in which three Syqe® cartridges comprising ground cannabis were subjected to a no-heat profile. THC and other cannabinoids were measured in the aerosol by Ultra-performance liquid chromatography (UPLC). Besides the control of the internal standard (Adrenosterone), the chromatogram showed an absence of all cannabinoids according to their limit of detection (LOD). In addition, the chromatogram was identical between all the tested three cartridges. As a positive control, a mixed solution that includes standards of 15 known cannabinoids (CBDV, CBDVA, THCV, CBD, CBDA, CBG, THCVA, CBN, CBGA, Δ9THC, Δ8THC, CBNA, CBC, THCA and CBCA) was tested. All the cannabinoids in the control mix were detected. Each cannabinoid was presented according to its retention time by the UPLC. The concentration of each cannabinoid was 10 μg/ml, and the detection was set to 228 nm except for CBCA that eluted at 254 nm.

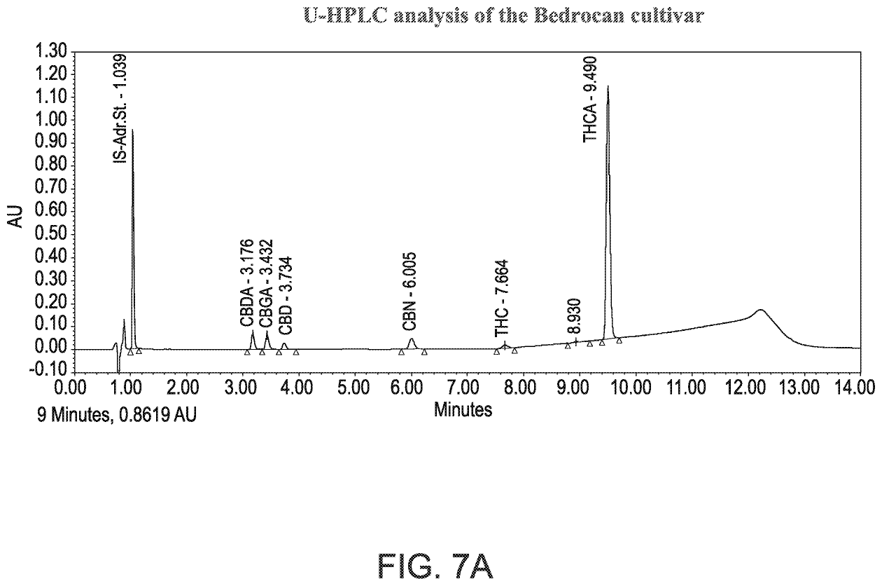

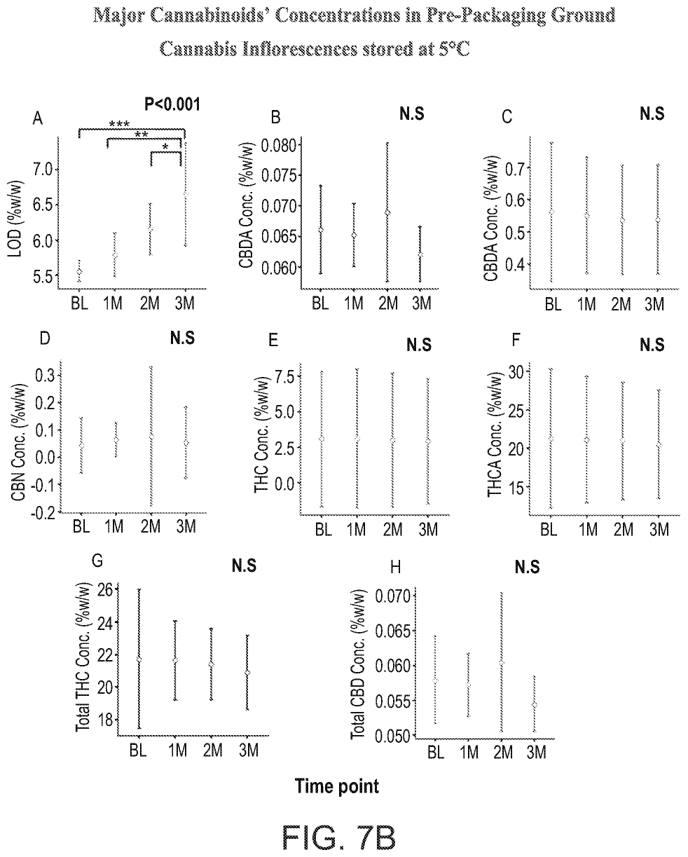

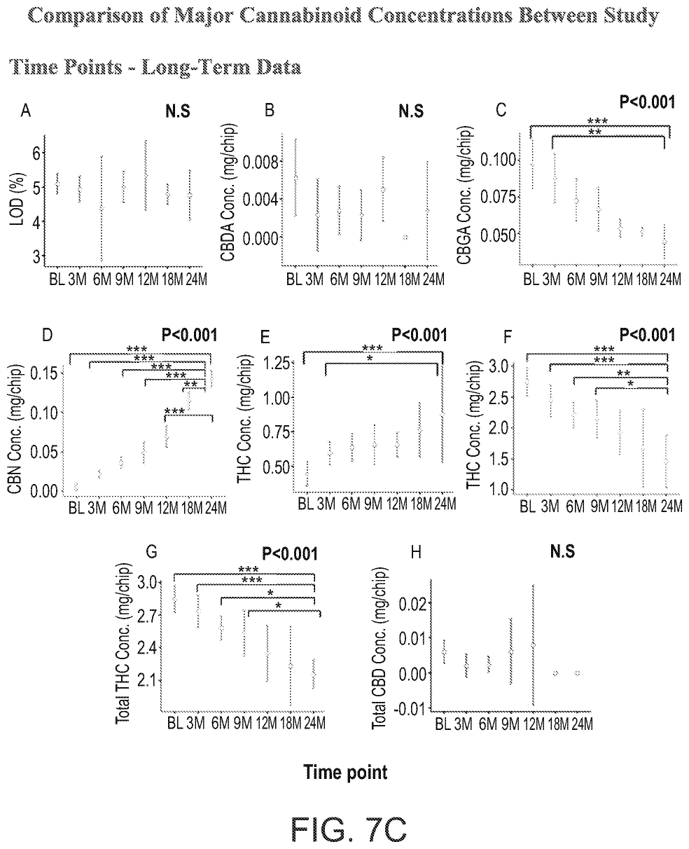

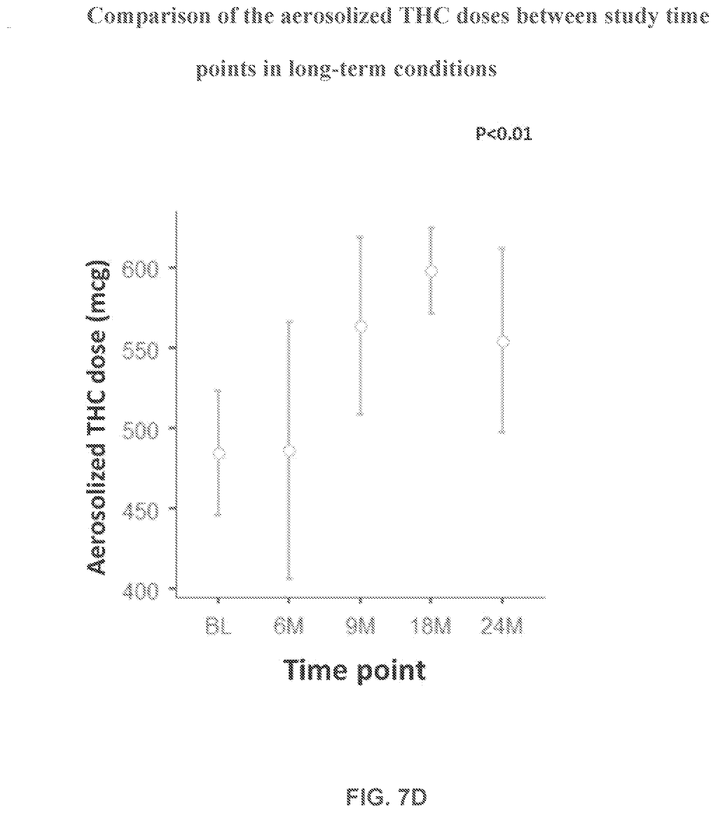

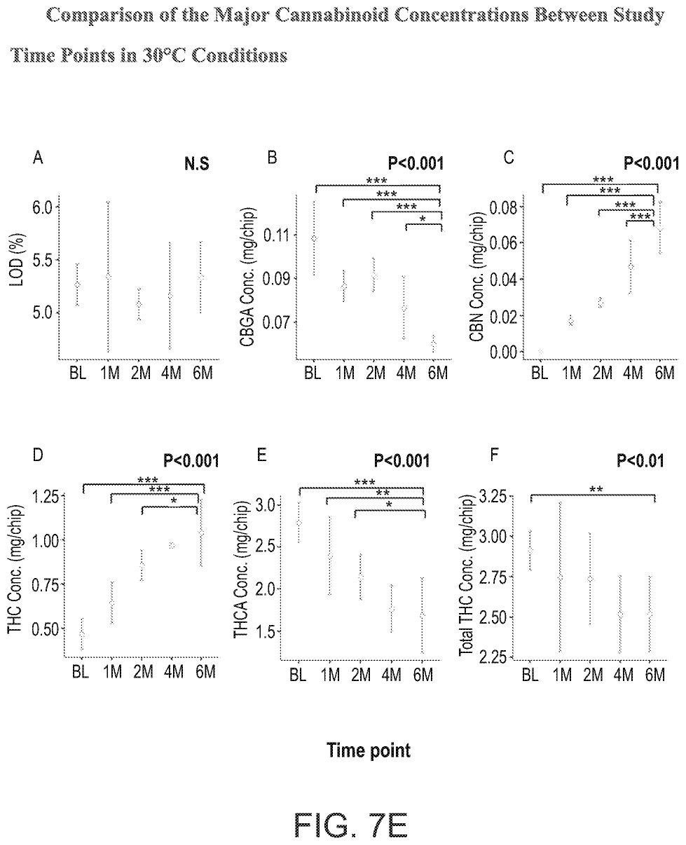

FIGS. 7A-E show results and analysis of an experiment performed by the inventors of this application in which changes in substance compositions were assessed for packaged cannabis stored over time under different environmental conditions, according to some embodiments. Background for the experiment performed in accordance with some embodiments: Although medical cannabis (MC) is gaining momentum worldwide, there is insufficient data regarding the long-term stability of phytocannabinoids in the plant material under different storage conditions. For vaporized cannabis, this means that there is insufficient data on the effect of varying storage conditions on the availability of (−)-A 9-trans-tetrahydrocannabinol (THC). The Syqe inhaler, a novel and precise means of cannabis delivery by inhalation, utilizes, in some cases, raw cannabis in ground form, pre-dosed and packaged in tamper proof cartridges. The aim of this experiment was to assess the stability of the phytocannabinoids in ground cannabis before and after packaging within Syqe cartridges under varying conditions as well as the stability of THC in the aerosolized dose, in accordance with some embodiments.

Methods: MC inflorescences were ground to a fine powder and the concentrations of the major phytocannabinoids therein were analyzed at different time points using ultra-high performance liquid chromatography (U-HPLC). Storage was done under different temperature and humidity conditions, before or after being packaged in Syqe cartridges. MC aerosolized by a the Syqe inhaler using Syqe cartridges stored for up to 2 years at 25° C. was analyzed for THC doses.
Results: Phytocannabinoids concentrations did not change under refrigeration conditions (up to 3 months storage). Under room temperature conditions (up to 2 years storage of cartridges), the absolute phytocannabinoid amount changed significantly, with some of the cannabinoids reducing and others increasing due for example to decarboxylation, however, the aerosolized Δ⁹-THC dose remained relatively stable throughout this period. Assessment of higher temperature and humidity conditions revealed a steeper change in phytocannabinoid concentrations within an even shorter period.
Conclusions: In the described study it was demonstrated that although significant changes in phytocannabinoid content occur during MC cartridge shelf life, the intended therapeutic Δ⁹-THC dose aerosolized by the Syqe inhaler remained stable. This may mean that MC in Syqe cartridges may be stored, for example, for at least two years after production at RT without affecting the Δ⁹-THC dose delivered to users by more than ±25%. Future studies are intended to address stability assessment of different cannabis cultivars within Syqe cartridges and the analysis of an extended profile of phytocannabinoids and the addition of terpenes.
Key words: Phytocannabinoids; Medical cannabis; Stability.

### LIST OF ABBREVIATIONS

THC—(−)-Δ⁹-trans-tetrahydrocannabinol
THCA—(−)-Δ⁹-trans-tetrahydrocannabinolic acid
CBD—Cannabidiol
CBDA—Cannabidiolic acid
U-HPLC—Ultra-high performance liquid chromatography
MC—Medical cannabis
CBGA—Cannabigerolic acid
CBN—Cannabinol
RH—Relative humidity
LOD—Loss on drying
VC—VaporChips
TR—Room temperature
CI—Confidence interval

### Introduction to the Performed Experiment

The interest of the Western medical field in the therapeutic potential of Cannabis sativa L. started following the report of O'Shaughnessy in his 1839 book on the therapeutic effects of the Indian hemp (1). Since then, despite a long period of cannabis prohibition (2), medical cannabis (MC) treatment is increasing worldwide for many clinical indications (3). Research on Cannabis sativa L. has advanced in recent decades and, thus far, 144 phytocannabinoids were identified (4). The main natural phytocannabinoids are (−)-Δ⁹-trans-tetrahydrocannabinolic acid (THCA), cannabidiolic acid (CBDA) and cannabigerolic acid (CBGA). These natural phytocannabinoids contain a carboxyl group (COOH). When heat is applied, they are converted by decarboxylation into (−)-Δ⁹-trans-tetrahydrocannabinol (THC), cannabidiol (CBD), and cannabigerol (CBG), respectively (5). Cannabinol (CBN) has been established as a marker for cannabis aging; it is created by THC oxidation and by cannabinolic acid (CBNA) decarboxylation. Based on THC and CBD concentration in cannabis cultivars (5), a categorization system has been implemented, with THC-rich cultivars designated as type I, CBD-rich cultivars as type III and cultivars with comparable concentrations of THC and CBD designated as type II (6). This categorization is currently the basis of most clinical treatment using the cannabis plant (7).

MC is mostly administered via smoking or vaporization (8,9). These administration routes are, in some cases, not metered or accurate (10). The Syqe inhaler (Syqe Medical Ltd.) was developed to administer clinically precise doses of phytocannabinoids by inhalation in a convenient and accurate manner from cannabis, such as cannabis stored in tamper proof cartridges manufactured under cleanroom conditions. In some embodiments, the cartridges contain vaporchips (VCs), each holding a precise amount a fine powder (11) of dried natural cannabis inflorescences, following a patented grinding method. In the described experiment, the user of the inhaler had no direct contact with the MC. Alternatively, in some embodiments, a user of the inhaler may have direct contact with the MC.

In some embodiments, a user can select a dose to be inhaled at the press of a button, and simply inhale via the mouthpiece. In the described experiment, THC was used as an indicator for dose selection (e.g., 500 μg THC) and for the phytocannabinoids, terpenes and other molecules of the whole inflorescence that are optionally vaporized along with the THC into the aerosol. Syqe technology was evaluated in several clinical trials (12-14), demonstrating good effectiveness, safety, and usability as well as narrow pharmacokinetic variability.

A recent publication demonstrated the effect of several temperatures at different processing stages on the concentrations of phytocannabinoids in herbal cannabis over a one year period (15). That study reported significant trends at room temperature (RT; 25° C.) for the stability of main phytocannabinoids. Hence, in the study described herein, one of the targets was to assess the stability of these phytocannabinoids during the manufacturing process at the Syqe Medical facility and within vacuum sealed cartridges for a period of two years. Other embodiments may include different storage time periods and/or different type(s) of packaging of the source material.

Another target was to assess whether storage time has an effect on Δ9-THC microgram doses aerosolized with a Syqe inhaler from such cartridges. The results were able to show that the aerosolized values are preserved within the specifications of a medical inhaler of delivering a predetermined dose ±25% (16).

### Methods

### Chemicals and Reagents

Liquid chromatography LiChrosolv® gradient grade acetonitrile, methanol, and water for the mobile phase; were purchased from Mercury Scientific and Industrial Products Ltd. (Rosh Haayin, Israel). LC/MS grade formic acid was purchased from BioLab Ltd. (Jerusalem, Israel). Phytocannabinoids analytical standards (>98%) THC, THCA, CBD, CBDA, CBN and CBGA were manufactured by Restek (Bellefonte, PA, USA)) and purchased from Silicol (Or Yehuda, Israel). Data was restricted to the requirements of the Israeli Ministry of health regulations.

### Medical Cannabis

Medical cannabis cultivar “Bedrocan” (Bedrocan, Netherlands) was used in all experiments, which strain contains about 22% THC and <1.0 CBD. It was free of pesticides, heavy metals (<0.2 ppm lead, <0.02 ppm mercury, and <0.02 ppm cadmium), stalks and foreign materials. Microbiological purity was confirmed (total aerobic microbial count of <10 colony forming units [CFU]/g, total yeast and mold count of <10 CFU/g, and absence of Pseudomonas aeruginosa, Staphylococcus Aureus, and bile-tolerant gram-negative bacteria).

### Experimental Design

### Syqe Inhaler & Cartridge

The Syqe inhaler 1.1 (Syqe Medical, Tel-Aviv, Israel) is, in accordance with some embodiments, a battery-operated, hand-held, thermal-selective dose-inhaler, which in some embodiments utilizes a tamper proof cartridge containing precise doses of processed medical grade cannabis powder, where, in some embodiments, each dose is packaged in a single-use VC. In some embodiments, the inhaler is designed to deliver vapor produced directly from the cannabis powder to an inhaling user. In some embodiments, THC is used as a precise marker for the delivered dose, which, in some cases, includes a full entourage of co-vaporized compounds. Additionally or alternatively, substance(s) other than THC are selectively vaporized, e.g. CBD, terpene(s) and/or other compounds, and may be used as a marker for the delivered dose.

In some embodiments, the Inhaler device has a plurality of settings that can be selected at a press of a button by the user to deliver, in accordance with some embodiments, one of a set of predefined doses using a corresponding heating protocol of the inhaler. In some embodiments, in operation, the inhaler heats the MC to a temperature below combustion and, in some embodiments, engages automatic thermal and/or airflow controls that ensure precise, accurate and high-efficiency delivery of the selected dose of medical cannabis produced aerosol to the user's lungs, potentially independent of the inhalation pattern of the individual user. In some embodiments, the device requires minimal training prior to use. In some embodiments, the device requires no direct contact with MC. In some embodiments, the device automatically generates logs of the inhalation process.

### MC Processing, Cartridge Production, Handling and Storage

In the described experiment, all MC handling was performed in a clean-room environment, from processing and until a cartridge is vacuum sealed, in accordance with some embodiments. In the described experiment, manufacture begun with the grinding of whole cannabis inflorescences to form a fine powder (particle diameter <1,000 μm). The powder was stored under refrigeration until its use in manufacturing VCs for packaging within Syqe cartridges.

In the described experiment, VCs were automatically manufactured by a precise dedicated machine, allotting, 13.5±0.5 mg of ground MC powder (Bedrocan strain) into each VC. Other embodiments may include different amounts of MC powder, for example ranging between 5-20 mg, 1-10 mg, 10-50 mg or intermediate, larger or smaller amounts of ground MC powder.

In the described experiment, sixty (60) VCs were packaged in each tamper proof cartridge. The Syqe inhaler used in the experiment released a single VC for each inhalation (Optionally, the VC is returned to the cartridge by the inhaler after use). In the described experiment, each VC was used by the inhaler only once, such as to ensure dose accuracy. Alternatively, in some embodiments, a VC may be used more than once.

In the described experiment, the cartridges were packaged in a vacuumed aluminum foil between production and use, thereby at least partially protected from light.

### Pre-Packaging Refrigeration

In the described experiment, Bedrocan strain MC powder was stored at 5° C.±2° C. with up to 12% relative humidity (RH) in a polypropylene container, sealed in an aluminum foil pouch (Oliver-Tolas Healthcare Packaging B.V.; Venray, Netherlands). In order to assess its stability during the handling and hold time, samples were taken from three separate growing batches and assayed at baseline and at 1, 2 and 3 months post grinding. Each sample was assayed for loss on drying (LOD), and of the concentrations of the major phytocannabinoids (CBDA, CBD, CBGA, CBN, THCA and THC).

### Post Packaging—Long-Term Data

In some embodiments, once cartridges are produced, they are stored, distributed and used at RT. In order to assess the shelf life of such cartridges, a long-term stability study was performed, during which cartridges were stored at 25° C.±2° C. (RH 60%±5%) in a calibrated and certified stability chamber (Memmert Constant climate chamber HPP260). 9, 6, 9, 8, 7, 4 and 4 separate growing batches were tested at baseline and after 3, 6, 9, 12, 18 and 24 months, respectively. At each time point samples of the ground inflorescence within the cartridges were analyzed for LOD and for the amount (mg) of each of the major phytocannabinoids (CBDA, CBD, CBGA, CBN, THCA and THC). Of the same batches, THC aerosolized by the Syqe inhaler was analyzed at baseline and at 6, 9, 18 and 24 months.

### Storage at 30° C.

Additionally, to assess the effects of a warmer temperature on MC, Syqe cartridges were stored post-production at an elevated temperature and humidity of 30° C.±2° C. (65% RH±5% RH). Three different growing batches were analyzed at baseline and at 1, 2, 4 and 6 months of storage.

### Chemical Analysis of Phytocannabinoids

Analysis of cannabis inflorescences for the main phytocannabinoids (CBDA, CBD, CBGA, CBN, THCA and THC) was performed as follows: 100 mg of ground Cannabis inflorescences were weighed in duplicate from one container and extracted with 25 mL methanol then sonicated for 40 min and centrifuged at 3000 rpm for 5 min. All samples were filtered through a 0.22 μm PP filter vail prior to analysis. The dilution and quantitation were preformed corresponding to calibration curves. The main six phytocannabinoids were analyzed by ultra-high-performance liquid chromatography (UHPLC: in your list of abbreviations you say U-HPLC, be consistent) system with a refrigerated auto-sampler, thermostatic column oven and ultraviolet detector, Waters ACQUITY UPLC H-Class. Chromatographic separation was achieved using UPLC column Waters Acquity C18, 1.7 μm particles, 2.1×150 mm maintained at 30° C. The phytocannabinoids were separated using a gradient elution with mobile phases of 0.1% (v/v) formic acid in double-distilled water (phase A) and acetonitrile (phase B), respectively. A constant flow rate of 0.4 mL/min was employed throughout. The gradient profile varied from 70% to 100% B in 10.5 min, held for 0.5 min in these conditions and then returned to the initial conditions.

The separation lasted 14 minutes.

For THC and CBD, the concentrations of the acid and its neutral counterpart were summed and reported as the total content. For example, the concentration of total THC (you already described the abbreviation for THC) was calculated as: Total THC=THCA*0.877+THC, with 0.877 being the molar ratio between the two compounds that corrects for a change in the mass of THCA as a result of the loss of CO2 (decarboxylation). This is shown in FIG. 7A: “U-HPLC analysis of the Bedrocan cultivar”.

### Treatment

In some embodiments, the Syqe inhaler is preprogrammed with several distinct heating protocols. In some embodiments, each heating protocol is tailored to heat a VC and deliver an aerosol containing 250, 500, 750 or 1,000 μg or intermediate, larger or smaller amounts of THC to the user. In some embodiments, THC serves as an indicator for dose selection and is optionally delivered with an entourage of phytocannabinoids, terpenes and other molecules of the whole inflorescence that vaporize into the aerosol with it. In this stability study, only the 500 μg Δ⁹-THC dose was assessed as it was found to be the optimal dose for chronic pain treatment in a previous study, with the best balance between pain reduction report and intoxication levels (14). Other dosage amounts are also to be contemplated, any may also serve as optimal dosing, depending on the need. For example, dosage amounts of between 50-5000 μg Δ⁹-THC, 200-1000 μg Δ⁹-THC, 50-500 μg Δ⁹-THC, 1000-5000 μg Δ⁹-THC, or intermediate, higher or lower ranges.

### Statistical Analysis

R software (V.1.1.463) with tidyverse (17) package was used to analyze changes in outcome measures by one way ANOVA test, using Fisher's exact test analysis. All analyses were followed by a Tukey post-hoc test for multiple comparison. Data are presented as Mean±95% confidence interval (CI). Differences were considered significant at the p<0.05 level.

### Results

### The Effect of Storage Parameters on the Phytocannabinoid Contents of Ground Cannabis Inflorescences (Refrigeration Conditions)

As depicted in FIG. 7B, while the increase of LOD percentage was found to be significant, no significant change was detected in any of the assayed phytocannabinoids. A one-way ANOVA, using Fisher's exact test analyses showed a significant increase in LOD from 5.6±0.06% at baseline (BL) to 6.6±0.29% at three months (F_{(3,8)}=21.56; p<0.001). Tukey post-hoc test showed significant difference between LOD levels at BL and at three months (t₍₈₎=−7.53; p<0.001), one month compared to three months (t₍₈₎=−5.93; p<0.01) and two months compared to three months (48)=−3.43; p<0.05). However, no significant change during the study period was found for, CBDA, CBGA, CBN, THC, THCA, Total THC and Total CBD (F_{(3,8)}=2.58; p=0.13, F_{(3,8)}=0.06; p=0.97, F_{(3,8)}=0.31; p=0.81, F_{(3,8)}=0.006; p=0.99, F_{(3,8)}=0.03; p=0.99, F_{(3,8)}=0.29; p=0.82 and F_{(3,8)}=2.59; p=0.12, respectively). As expected, due to the baseline concentrations of CBD in this cultivar (<1%), CBD concentration were mostly undetected at all time points. These results confirm that the conditions in which the ground inflorescences were preserved in the described experiment maintain stable phytocannabinoid concentrations.

### The Effect of Storage Parameters on the Phytocannabinoid Contents of Ground Cannabis Inflorescences Packaged in Syqe Cartridges (Long-Term Data)

In order to assess the effect of room temperature storage on the phytocannabinoids content of ground cannabis in Syqe cartridges, a two-year stability study was performed.

As shown in FIG. 7C, one way ANOVA, using Fisher's exact test analyses demonstrated no significant change in LOD levels and in CBDA and Total CBD amounts (F_{(6,31)}=0.68; p=0.66, F_{(6,40)}=1.90; p=0.10 and F_{(6,37)}=0.67; p=0.67, respectively). Nonetheless, CBGA, THCA, and Total THC amounts decreased significantly (F_{(6,40)}=8.96; p<0.001, F_{(6,40)}=11.26; p<0.001 and F_{(6,40)}=9.94; p<0.001), Tukey post-hoc test showed a significant decrease of CBGA amounts between BL and 24 months and between three months and 24 months (t₍₄₀₎=5.33; p<0.001 and t₍₄₀₎=−4.07; p<0.01, respectively). THCA decreased significantly between BL and 24 months (t₍₄₀₎=6.72; p<0.001), three months and 24 months (t₍₄₀₎=4.79; p<0.001), six months and 24 months (t₍₄₀₎=3.96; p<0.01) and between nine months and 24 months (t₍₄₀₎=3.54; p<0.05). Total Δ⁹-THC decreased significantly between BL and 24 months (t₍₄₀₎=5.83; p<0.001), three months and 24 months (t₍₄₀₎=4.55; p<0.001), six months and 24 months (t₍₄₀₎=3.56; p<0.05) and between nine months and 24 months (t₍₄₀₎=3.17; p<0.05).

CBN and Δ⁹-THC amounts increased significantly (F_{(6,40)}=122.18; p<0.001 and F_{(6,40)}=6.04; p<0.001). Notably, CBN amounts increased between BL and 24 months (t₍₄₀₎=−22.18; p<0.001), three months and 24 months (t₍₄₀₎=−18.17; p<0.001), six months and 24 months (t₍₄₀₎=−17.08; p<0.001), nine months and 24 months (t₍₄₀₎=−14.82; p<0.001), twelve months and 24 months (t₍₄₀₎=−11.40; p<0.001) and between 18 months and 24 months (t₍₄₀₎=−3.84; p<0.01). THC amounts increased between BL and 24 months (t₍₄₀₎=−5.40; p<0.001) and between three months and 24 months (t₍₄₀₎=−3.29; p<0.05). Expectedly, CBD concentration were mostly non-detected at all time points, as the pre-storage amount of its precursor, CBDA, was 0.006 mg/sample or less.

### Use of Stored Cannabis Cartridges in the Syqe Inhaler, in Accordance with Some Embodiments

In the framework of the two-year stability study, samples were also taken for aerosolization using the Syqe Inhaler. The Syqe Inhaler was set to aerosolize 500 μg THC doses, in accordance with some embodiments. Data is provided only for time points in which three or more batches were tested (namely, BL and 6, 9, 18 and 24 months). As shown in FIG. 7D, contrary to the changes in phytocannabinoid content of the ground inflorescences inside the cartridges, aerosolized THC doses remained relatively stable, but with some statistically significant change over time (F_{(4,15)}=9.09; p<0.001) by one-way ANOVA, using Fisher's exact test analysis. BL, 6-, 9-, 18-, and 24-months' values were 484.76±31.28n, 486.30±50.56n, 563±22.06 μg, 597.94±16.75 μg and 554.67±36.00 μg, respectively. All aerosolized THC doses were well within the 500 μg±25% range (i.e., 375-625 μg) that apply to pharmaceutical grade inhalers (16). Tukey post-hoc test did not show any significant change between the time points.

### The Effect of Storage Parameters on the Phytocannabinoid Contents of Ground Cannabis Inflorescences Packaged in Syqe Cartridges, in Accordance with Some Embodiments (Storage at Elevated Temp of 30° C.)

In the following stability study, aimed at storage conditions harsher than room temperature, namely an elevated temperature of 30° C., using one-way ANOVA, with Fisher's exact test analyses, it was shown that LOD values did not vary significantly over time (F_{(4,12)}=1.12; p=0.19), possibly due to being packed within the cartridge and held in VCs. However, CBGA, THCA and Total THC decreased significantly (F_{(4,12)}=33.57; p<0.001, F_{(4,12)}=22.52; p<0.001 and F_{(4,12})=6.46; p<0.01, respectively). Tukey post-hoc test showed a significant decrease in CBGA amounts between BL and six months (t₍₁₂₎=11.17; p<0.001), one month and six months (t₍₁₂₎=5.68; p<0.001), two months and six months (t₍₁₂₎=6.76; p<0.001) and between four months and six months (t₍₁₂₎=3.55; p<0.05). There was a significant decrease of THCA amounts between BL and six months (t₍₁₂₎=8.39; p<0.001), one month and six months (t₍₁₂₎=5.00; p<0.01) and between two months and six months (t₍₁₂₎=3.21; p<0.05). Total THC levels decreased significantly between BL and six months (t₍₁₂₎=4.36; p<0.01). Additionally, CBN and THC increased significantly (F_{(4,12)}=106.72; p<0.001 and F_{(4,12)}=44.44; p<0.001, respectively). Tukey post-hoc test showed a significant increase in CBN amounts between BL and six months (t₍₁₂₎=−19.26; p<0.001), one month and six months (t₍₁₂₎=−13.30; p<0.001), two months and six months (t₍₁₂₎=−10.79; p<0.001) and between four months and six months (t₍₁₂₎=−5.66; p<0.001). Δ⁹-THC levels increased between BL and six months (t₍₁₂₎=−11.81; p<0.001), one month and six months (t₍₁₂₎=−7.52; p<0.001) and between two months and six months (t₍₁₂₎=−3.51; p<0.05) (see FIG. 7E). As expected, CBD, CBDA and Total CBD amounts were mostly undetected at all time points and could not be analyzed.

### DISCUSSION

In the study described herein, no significant changes were found in the main phytocannabinoid content of ground cannabis after 3 months' bulk storage at 5° C. in a polypropylene container and sealed in aluminum foil pouch. This potentially suggests that grinding of the bulk product may be performed at least up to 3 months prior to cartridge manufacture, optionally without a detectable effect on cannabinoid composition of the ground inflorescences.

On the other hand, during two years of storage of final product, packaged in cartridges at room temperature (25° C.), significant trends in the main phytocannabinoids concentrations in the ground inflorescences within the cartridges were observed. Specifically, CBGA, THCA and Total THC concentrations decreased, while CBN and THC concentrations increased. These findings are essentially in line with Milay et al., (2020) who reported that a THC-rich cultivar stored for one ear at RT demonstrated THC and CBN concentration increase reflecting THC generation due to decarboxylation of THCA by heat and light and THC loss by degradation to CBN (15).

The loss of THCA and Total THC in the ground inflorescences within the vacuumed sealed cartridges was expected to be correlated with a respective decrease in the aerosolized THC doses, however, aerosolized THC doses remained within the 500 μg±25% range (i.e., 375-625 μg) as required for pharmaceutical grade inhalers (16). This apparent discrepancy may be potentially explained by the fact that the amount of Δ⁹-THC increased and so did the proportion of THC/THCA. It is possible that while the Syqe Inhaler, in some of its configurations, causes the vaporization and decarboxylation of THCA to deliver THC, other configurations may include vaporizing and providing THC directly from the plant material, with no need for decarboxylation. Therefore, during intermediate storage duration and conditions, while THCA degrades to THC by the natural occurrence of decarboxylation, the inhaler may still deliver the desired amount of THC directly from the plant. This natural degradation is enhanced by heating during use of the inhaler. The heating, as well as other operational features, such as airflow, may be adjusted to compensate for the degradation of the THCA. Potentially, if the efficiency of THC vaporization is greater than that of the vaporization and decarboxylation of THCA, the increase in THC during storage at 25° C. might compensate for the concomitant loss of THCA and even total THC.

The study design under elevated temperature (30° C.) demonstrated a more rapid degradation and decarboxylation processes to an extent that would potentially shorten the shelf life of the product. Therefore, in some cases, such temperatures should be avoided in the chain of pharmaceutical grade cannabis production, storage, and transport, and optionally users should be encouraged to maintain the cartridges at lower temperatures if possible.

Based on this study, it is suggested that storage at 5° C. suffices at least for up to 3 months for MC powder packaged as detailed in this study. Accordingly, freezing storage may be avoided for this phase of cartridge manufacture, thereby potentially simplifying logistics of the production process and reducing its costs. Once a cartridge is produced, it may be maintained at RT or a lower temperature to maintain a shelf life of at least 2 years potentially without a significant effect of the dose delivered by aerosolization. Accordingly, in some embodiments, refrigeration may be unnecessary at this stage, potentially further simplifying distribution and storage for the entire supply chain, as well as being convenient to users. In some embodiments, significant exposure of cartridges to a temperature significantly higher than 25° C. should be avoided, as shown for example by the described experiment for storage at 30° C.

### CONCLUSIONS

In the described study it was demonstrated that although significant trends may occur during an extended duration of the cartridges “shelf life”, the pronounced therapeutic aerosolized THC dose potentially remains relatively stable, within a dosage range of 500 μg±25% range, in accordance with some embodiments. These findings may provide an important improvement to the field of medical cannabis, in which physicians and users commonly lack knowledge of the aerosolized doses, for example when using devices having no precision level or having a lower precision level than the Syqe inhaler; for example when cannabis inflorescences are smoked in a cigarette, with or without a tobacco additive. Future studies should include the stability assessment of different cannabis cultivars within the Syqe Cartridges and the analysis of an extended profile of all phytocannabinoids and the addition of terpenes.

### REFERENCES PERTAINING TO THE EXPERIMENT DESCRIBED HEREINABOVE

1. O'Shaughnessy WB. Case of Tetanus, Cured by a Preparation of Hemp (the Cannabis indica). In: O'Shaughnessy WB, editor. Transactions of the Medical and Physical Society of Bengal, On the preparations of Indian hemp. Calcutta Bishops Cotton Press; 1839. p. 1838-40.
2. David B-T, Blickman T, Jelsma M. The rise and decline of cannabis prohibition. Amsterdam/Swansea: Global Drug Policy Observatory/Transnational Institute; 2014.
3. Boehnke K F, Litinas E, Clauw D J. Medical cannabis associated with decreased opiate medication use in retrospective cross-sectional survey of chronic pain users. J Pain. 2016; 17(6):739-44.
4. Berman P, Futoran K, Lewitus G M, Mukha D, Benami M, Shlomi T, et al. A new ESI-LC/MS approach for comprehensive metabolic profiling of phytocannabinoids in Cannabis. Sci Rep. 2018; 8(1):1-15.
5. Peschel W. Quality control of traditional Cannabis tinctures: Pattern, markers, and stability. Sci Pharm. 2016; 84(3):567-84.
6. Russo E B. Taming THC: potential cannabis synergy and phytocannabinoid-terpenoid entourage effects. Br J Pharmacol. 2011; 163(7):1344-64.
7. Aviram J, Lewitus G M, Vysotski Y, Uribayev A, Procaccia S, Cohen I, et al. Short-Term Medical Cannabis Treatment Regimens Produced Beneficial Effects among Palliative Cancer Users. Pharmaceuticals. 2020; 13(12):435.
8. Hazekamp A, Ware M A, Muller-Vahl K R, Abrams D, Grotenhermen F. The Medicinal Use of Cannabis and Cannabinoids—An International Cross-Sectional Survey on Administration Forms. J Psychoactive Drugs. 2013; 45(3):199-210.
9. Aviram J, Pud D, Gershoni T, Schiff-Keren B, Ogintz M, Vulfsons S, et al. Medical Cannabis Treatment for Chronic Pain: Outcomes and Prediction of Response. Eur J Pain. 2020; 25(2):359-74.
10. Huestis M A. Human Cannabinoid Pharmacokinetics. Chem Biodivers. 2009; 4(8):1770-804.
11. Lai W, Lu W, Chen C. The new expression of the effectiveness of powder classification. Adv Powder Technol [Internet]. 2005; 16(6):611-20. Available from: www(dot)dx(dot)doi(dot)org/10.1163/156855205774483352
12. Eisenberg E, Ogintz M, Almog S. The Pharmacokinetics, Efficacy, Safety, and Ease of Use of a Novel Portable Metered-Dose Cannabis Inhaler in Users With Chronic Neuropathic Pain: A Phase 1a Study. J Pain Palliat Care Pharmacother. 2014; 28(3):216-25.
13. Vulfsons S, Ognitz M, Bar-Sela G, Raz-Pasteur A, Eisenberg E. Cannabis treatment in hospitalized users using the SYQE inhaler: Results of a pilot open-label study. Palliat Support Care. 2020; 18(1):12-7.
14. Almog S, Aharon-Peretz J, Vulfsons S, Ogintz M, Abalia H, Lupo T, et al. The Pharmacokinetics, Efficacy, and Safety of a Novel Selective-Dose Cannabis Inhaler in Users with Chronic Pain: A Randomized, Double-Blinded, Placebo-Controlled Trial. Eur J Pain [Internet]. 2020 May 23 [cited 2020 May 29]; 24(8):1505-16. Available from: www(dot)onlinelibrary(dot)wiley(dot)com/doi/abs/10(dot)1002/ejp(dot)1605
15. Milay L, Berman P, Shapira A, Guberman O, Meiri D. Metabolic Profiling of Cannabis Secondary Metabolites for Evaluation of Optimal Postharvest Storage Conditions. Front Plant Sci. 2020; 11(October):1-15.
16. The United States Pharmacopeial Convention. Metered-dose inhalers and dry powder inhalers. 2012.
17. Wickham H. Tidyverse: Easily install and load ‘Tidyverse” packages. Version 1.3.0 [Internet]. Comprehensive R Archive Network (CRAN). 2019. Available from (www(dot)cran(dot)r-project(dot)org/web/packages/tidyverse/tidyverse(dot)pdf

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features, which are, for clarity, described in the context of separate embodiments, may also be provided in combination in a single embodiment. Conversely, various features, which are, for brevity, described in the context of a single embodiment, may also be provided separately or in any suitable subcombination or as suitable in any other described embodiment. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of delivering via inhalation from a same source material both pharmacologically-effective doses and placebo doses released from an inhalation device, comprising:
selecting a type of dose to be delivered via an inhalation device, the type of dose being a pharmacologically-effective dose or a placebo dose;
based on the type of dose, selecting at least one of a heating profile and an airflow profile to be applied to the source material;
releasing from the source material one or more substances of a type and/or amount which is either pharmacologically effective or psychologically effective, depending on the type of dose to be delivered.

2. The method according to claim 1, wherein the placebo dose comprises substances having a taste and/or scent similar or close to those of substances released for a pharmacologically-effective dose.

3. The method according to claim 1, wherein the placebo dose further comprises the release of vapor.

4. The method according to claim 1, wherein releasing a placebo dose includes releasing one or more sensory substances from the source material, while avoiding or reducing release of active substances from the source material.

5. The method according to claim 1, wherein the source material comprises cannabis, wherein the one or more sensory substances include terpenes and the active substances include THC.

6. The method according to claim 1, wherein selecting of a heating profile comprises selecting a temperature range which is low enough so that only the one or more sensory substances are vaporized from the source material, while one or more active substances do not reach a vaporization state.

7. The method according to claim 1, further comprising tracking conditions associated with storage of the source material over time, and wherein selecting is performed at least partially based on the tracked conditions.

8. A method of delivering via inhalation at least one placebo dose from a THC-carrying material, comprising vaporizing at least one component of the THC-carrying material, wherein said at least one component is not THC; wherein said THC-carrying material is at a temperature below 130° C.

9. The method according to claim 8, wherein said at least one component comprises a terpene.

10. The method of claim 8, wherein said THC-carrying material is at a temperature below 50° C.

11. The method of claim 8, wherein said THC-carrying material is at room temperature.

12. A method of delivering via inhalation one or more substances released from a source material, the one or more substances having a selected sensory profile which is experienced by an inhaling user, the method comprising:
controlling heating of the source material to release the one or more substances from the source material, wherein the source material is maintained at a temperature below 130° C.; and
delivering the released one or more substances to the inhaling user such that the user experiences the selected sensory profile.

13. The method according to claim 12, wherein the sensory profile comprises a flavor and a scent.

14. The method according to claim 12, wherein the sensory profile further comprises vapor related sensations or appearance of vapor.

15. The method according to claim 12, wherein the one or more substances are psychologically effective and not pharmacologically effective and wherein the selected sensory profile is similar to that experienced when one or more pharmacologically effective substances are released from the same source material.

16. The method according to claim 15, wherein the one more psychologically effective substances include terpenes and the one more pharmacologically effective substances include cannabinoids.

17. An inhaler for delivery of types of doses, including pharmacologically effective dose and a placebo dose, to a user by inhalation from the same source material, the inhaler comprising:
an airflow conduit for conducting airflow to a proximal opening of a mouthpiece;
a holder configured to position the source material at a delivery position within the airflow conduit; and
a circuitry programmed to apply heating and/or airflow profiles to the source material suitable for the delivery of the selected type of dose.

18. The inhaler of claim 17, wherein said circuitry is preprogrammed for a placebo dose with heating to a temperature of less than 130° C.

19. The inhaler of claim 17, wherein said circuitry is preprogrammed for a placebo dose with no heating, and having an airflow profile suitable to release selected substance(s) at selected amount(s) according to a delivery regimen.
